(19) United States
(12) Patent Application Publication
(10) Pub. No.: US 2010/0291086 A1
(43) Pub. Date: Nov. 18, 2010

(54) USE OF ESTROGEN AND ANDROGEN BINDING PROTEINS IN METHODS AND COMPOSITIONS FOR TREATING GYNAECOLOGICAL CANCERS

(76) Inventors: Christopher Hovens, Surrey Hills (AU); Niall Corcoran, Flemington (AU); Anthony Costello, Port Melbourne (AU)

Correspondence Address: WINSTON & STRAWN LLP;PATENT DEPARTMENT, 1700 K STREET, N.W., WASHINGTON, DC, 20006, US

(21) Appl. No.: 12/677,694

(22) PCT Filed: Sep. 10, 2008
(86) PCT No.: PCT/AU08/01338

§ 371 Date: May 25, 2010

Related U.S. Application Data
(60) Provisional application No. 60/971,821, filed on Sep. 12, 2007.

(30) Foreign Application Priority Data
Sep. 11, 2007 (AU) 2007904940

(51) Int. Cl. A61K 39/395; C07K 16/26; C07K 2/00; C07K 14/00; C07H 21/04; C12N 15/63; A61K 38/17; A61K 31/7088; A61P 35/00; A61P 5/28; A61P 5/32

(52) U.S. Cl. 424/135.1; 530/387.3; 530/388.24; 530/389.2; 530/300; 530/402; 536/23.5; 435/320.1; 514/10.2; 514/44 R; 424/141.1; 424/130.1

(57) ABSTRACT

The present invention provides a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased. The invention also provides for the treatment or prevention of cancers such as ovarian cancer, breast cancer and endometrial cancer using the polypeptides.

### FIELD OF THE INVENTION

The present invention relates generally to the field of oncology, and more particularly to the use of polypeptides in the prevention or treatment of cancers of the breast, ovary and endometrium.

### BACKGROUND TO THE INVENTION

Breast cancer is the most-frequently diagnosed cancer and the second most common cause of death from cancer in women, exceeded only by lung cancer. Breast cancer is a disease causing significant morbidity and mortality throughout the world. There are many different types of breast cancer, and it is not uncommon for a single breast tumor to be a combination of types and to have a mixture of invasive and in situ cancer (cancer that has not spread nor invaded surrounding tissue, and remains confined to the ducts or lobules of the breast).

The two main types of breast adenocarcinomas are ductal carcinomas (also known as intraductal carcinoma), which is the most common non-invasive breast cancer, and lobular carcinomas. Ductal carcinoma in situ (also known as intraductal carcinoma) is the most common type of noninvasive breast cancer. Lobular carcinoma in situ (LCIS, also called lobular neoplasia), while not regarded as a true cancer, is sometimes classified as a type of noninvasive breast cancer, and women with this condition have a higher risk of developing an invasive breast cancer.

The most common breast cancer is invasive (or infiltrating) ductal carcinoma (IDC)-about 80% of invasive breast cancers are IDC. This cancer originates in a duct of the breast, and has progressed past the wall of the duct and invaded the fatty tissue of the breast. At this point, it can metastasize, or spread to other parts of the body via the lymphatic system and bloodstream. About 10% of invasive breast cancers are invasive (or infiltrating) lobular carcinoma (ILC), which starts in the lobules of the breast, which can metastasize to other parts of the body.

In addition to the above breast cancers, there are uncommon types of breast cancer such as inflammatory breast cancer and medullary cancer, which account for about 1-3% and 5% of all of breast cancers, respectively, metaplastic tumors and tubular carcinomas (both rare variants of invasive ductal cancer), mucinous carcinoma (also known as colloid carcinoma), Paget disease of the nipple, phylloides tumor, and tubular carcinoma.

Women living in Australia, North America and Western Europe have the highest rates of breast cancer in the world. The chance of developing invasive breast cancer at some time in a woman's life is about 1 in 8 (13% of women). World-wide, about 1,150,000 people are diagnosed with breast cancer each year, and of those diagnosed about 410,000 die each year, In Australia, 11,866 new cases were diagnosed in 2001, with the incidence rising from 100.4 cases per 100,000 population in 1991 to 117.2 cases per 100,000 population in 2001. Furthermore, it is estimated that in 2007 about 178,480 new cases of invasive breast cancer will be diagnosed among women in the United States.

In Australia, about 1 in 70 women will develop ovarian cancer during their lifetime—every year around 1200 women will be diagnosed with ovarian cancer and nearly 800 women will die of the disease. Ovarian cancer is the sixth most common cause of cancer death in women—in Australia it is now more common than cervical cancer and it kills many more women. Of the 1200 cases diagnosed each year, about 75% will be advanced stage, and a staggering 75% will not survive past 5 years. In the United States, ovarian cancer is the leading cause of death from gynecologic malignancies and is the fourth most common cause of cancer mortality in women. During 2006, there were projected to be over 20,180 new cases of ovarian cancer in the US resulting in 15,310 deaths (as estimated by the American Cancer Society).

Given the prevalence and seriousness of these diseases, significant research has been directed to achieving control or cures for breast and ovarian cancer. There are a number of treatments known in the art, all of which have at least one adverse side effect.

For breast cancer, primary treatment is surgery for most patients, often with combined with radiation therapy. Chemotherapy, hormone therapy, or both may also be used, depending on tumor and patient characteristics. For inflammatory or advanced breast cancer, primary treatment is systemic therapy, which, for inflammatory breast cancer, is usually followed by surgery and radiation therapy. Surgery is usually not helpful for advanced cancer. Paget's disease of the nipple is treated as for other forms of breast cancer, although a very few patients can be treated successfully with local excision only.

Localised therapies are intended to treat a tumor at the site without affecting the rest of the body, and include surgery and radiation therapy. Mastectomy, championed by William Halstead more than 100 years ago has saved the lives of millions of women with advanced breast cancer, and involves removal of the entire breast, (or both breasts). Radical mastectomy, which involved removal of the breast, axillary lymph nodes and the pectoral muscles, has largely been replaced by a less-disfiguring approach, known as modified radical mastectomy, which involves removal of the axillary nodes and the breast.

The complications of such radical surgery have resulted in the push towards alternative treatments that do not involve loss of the breast. In the 1980s, breast-conserving surgery (BCS) with a 6-week protracted course of whole-breast irradiation (WBI) became popular. In breast conserving surgery, removal of only the breast lump and a surrounding margin of normal tissue is conducted in lumpectomy, and radiation therapy and/or chemotherapy may be conducted subsequent to surgery. Partial (or segmental) mastectomy (often referred to as quadrantectomy) removes more breast tissue (up to a quarter of the breast) than a lumpectomy (up to one-quarter of the breast). Similarly, radiation therapy and/or chemotherapy is usually given after surgery.

Possible side effects of mastectomy and lumpectomy include wound infection, hematoma (accumulation of blood in the wound), and seroma (accumulation of clear fluid in the wound). If axillary lymph nodes are also removed, swelling of the arm (lymphedema) is common—about 25% to 30% of women who had underarm lymph nodes removed develop lymphedema. Lymphedema also occurs in up to 5% of women who have sentinel lymph node biopsy; a surgical breast cancer treatment involving removing the sentinel node (the first lymph node into which a tumor drains) and establishing whether further lymph nodes need to be surgically removed. This swelling may last for only a few weeks but may also be long lasting. Other side effects of surgery include temporary or permanent limitations in arm and shoulder movement, numbness of the upper-inner arm skin, tenderness of the area, and hardness due to scar tissue that forms in the surgical site. If upon lumpectomy there is cancer at the margin of biopsied tissue, additional surgery (re-excision) may be required to remove further tissue.

External beam radiation therapy, treatment with high-energy rays or particles that destroy cancer cells, may be used to destroy cancer cells that remain in the breast, chest wall, or underarm area after surgery. The area treated by radiation therapy may also include supraclavicular lymph nodes (nodes above the collarbone) and internal mammary lymph nodes (nodes beneath the sternum or breast bone in the center of the chest). More recently, a new paradigm of partial-breast treatment with breast conserving surgery and partial-breast irradiation (PBI) has been proposed which administers radiation over a much shorter period, and to only the part of the breast with the cancer. It is hoped that partial breast irradiation, which is currently being done in clinical research trials, will prove to be equal to the current, standard whole breast irradiation. Nonetheless, the complications of external beam radiation therapy are swelling and heaviness in the breast, sunburn-like skin changes in the treated area which can last for 6 to 12 months, and fatigue. A further, albeit rare, complication is the development of another cancer called angiosarcoma, which can be treated with mastectomy but can be fatal. Brachytherapy, also known as internal or interstitial radiation, involves the placement of radioactive seeds or pellets directly into breast tissue next to the cancer. Another form of brachytherapy, MammoSite, consists of a balloon attached to a thin tube which is inserted into the lumpectomy space and filled with a saline solution into which a radioactive source is then temporarily placed (through the tube), and following treatment the balloon is then deflated and removed. Complications of brachytherapy include seroma, balloon rupture and wound infections.

Following axillary dissection or radiation therapy, lymphatic drainage of the ipsilateral arm can be impaired, sometimes resulting in significant swelling due to lymphedema. The magnitude of this effect may be proportional to the number of nodes removed. A specially trained therapist must treat lymphedema—special massage techniques once or twice daily may help drain fluid from congested areas toward functioning lymph basins; low-stretch bandaging is applied immediately after manual drainage. After the lymphedema resolves, patients require daily exercise and overnight bandaging of the affected limb indefinitely.

In most cases, chemotherapy is most effective, either as an adjuvant or neoadjuvant therapy, when combinations of more than one chemotherapy drug are used together. The most effective cytotoxic drugs for treatment of metastatic breast cancer are capecitabine, doxorubicin (including its liposomal formulation), gemcitabine, the taxanes paclitaxel and docetaxel, and vinorelbine. Response rate to a combination of drugs is higher than that to a single drug, but survival is not improved and toxicity is increased. Thus, some oncologists use single drugs sequentially. Combination chemotherapy regimens (eg, cyclophosphamide, methotrexate, plus 5-fluorouracil doxorubicin, plus cyclophosphamide) are more effective than a single drug. Acute adverse effects depend on the regimen, but usually include nausea, vomiting, mucositis, fatigue, alopecia, myelosuppression, and thrombocytopenia. The most commonly used combinations include; Cyclophosphamide (Cytoxan), methotrexate (Amethopterin, Mexate, Folex), and fluorouracil (Fluorouracil, 5-FU, Adrucil) [abbreviated CMF]; Cyclophosphamide, doxorubicin (Adriamycin), and fluorouracil [abbreviated CAF]; Doxorubicin (Adriamycin) and cyclophosphamide [abbreviated AC]; Doxorubicin (Adriamycin) and cyclophosphamide followed by paclitaxel (Taxol) or docetaxel (Taxotere) [abbreviated AC-->T] or docetaxel concurrent with AC [abbreviated TAC]; Doxorubicin (Adriamycin), followed by CMF; Cyclophosphamide, epirubicin (Ellence), and fluorouracil [abbreviated CEF] with or without docetaxel; Cyclophosphamide and Docetaxel (TC); and Gemcitabine (Gemzar) and paclitaxel (Taxol) [abbreviated GT].

These drugs often have severe toxicity and their use often requires close supervision. For instance, the complications of cyclophosphamide therapy can include aemorrhagic cystitis; gonadal suppression; pigmentation, rash; cardiotoxicity; fluid retention; poor wound healing; hyperuricaemia; gastrointestinal upset; nephrotoxicity; hepatotoxicity; pulmonary fibrosis; sec malignancy, infection; alopecia; haematological effects; and veno-occlusive disease.

The complications of methotrexate therapy can include CNS toxicity; hepato- and nephro-toxicity; gastrointestinal toxicity including ulcerative stomatitis; bone marrow depression; immunosuppression; opportunistic infection especially P. carinii pneumonia; lymphatic, proliferative disorders; fatigue, malaise; infertility; pulmonary toxicity; rash; fever; cardiovascular, and ophthalmic effects.

The complications of fluorouracil therapy can include local pain, pruritus; pigmentation, burning, dermatitis, and scarring.

The complications of doxorubicin therapy can include cardiotoxicity, mucositis; myelosuppression, leucopenia, haemorrhage; injection site reaction; red urine; male infertility; premature menopause; thromboembolism; alopecia; anorexia; gastrointestinal upset, abdominal pain; hyperpigmentation; dehydration; and flushing.

The complications of docetaxel therapy can include rash, sensitivity phenomena; alopecia; hand foot syndrome; haematological effects; oedema; gastrointestinal upset; hypertension, hypotension; neurosensory symptoms; injection site reaction; lacrimation both with and without conjunctivitis; visual effects; ear, and labyrinth disorders.

The complications of epirubicin therapy can include cardiotoxicity; extravasation; vesication; myelosuppression; CNS, cardiovascular, haematological, gastrointestinal, ocular, hepatic disturbances; dehydration; alopecia; hyperuricaemia; red urine; thromboembolism; amenorrhoea, and premature menopause.

The complications of gemcitabine therapy can include flu-like syndrome; oedema; hepatic, cardiac, blood disorders; somnolence; gastrointestinal upset; pulmonary effects; proteinuria, haematuria; rash (severe skin reactions, rare); pruritus; alopecia; and mouth ulceration.

The complications of taxol therapy can include hypersensitivity including anaphylactoid reactions; cardiovascular effects incl hypotension, arrhythmia; bone marrow suppression; peripheral neuropathy; arthralgia, myalgia; raised LFTs; gastrointestinal upset, perforation; alopecia; and injection site reactions.

A problem of multi-targeted agents is that the clinical effects of these drugs most likely result from both their on-target, and off target, effects. The toxicities mentioned above can be off-target effects, resulting from unintended and unknown functions, however it has been proposed that clinicians prefer multi-targeted drugs since they aim to maximize the chance for antitumor activity. Changes in dose (to increase efficacy) may amplify these off-target effects.

Choice of therapy depends on the hormone-receptor status of the tumor, length of the disease-free interval (from diagnosis to manifestation of metastases), number of metastatic sites and organs affected, and patient's menopausal status. Most patients with symptomatic metastatic disease are treated with systemic hormone therapy or chemotherapy. Radiation therapy alone may be used to treat isolated, symptomatic bone lesions or local skin recurrences not amenable to surgical resection. Radiation therapy is the most effective treatment for brain metastases, occasionally achieving long-term control. Patients with multiple metastatic sites outside the CNS should initially be given systemic therapy. There is no proof that treatment of asymptomatic metastases substantially increases survival, and it may reduce quality of life.

Hormone therapy is another form of adjuvant systemic therapy. The hormone estrogen is produced mainly by a woman's ovaries until menopause, after which it is made mostly in the body's fat tissue where a testosterone-like hormone (androstenedione) made by the adrenal gland is converted into estrogen by the enzyme aromatase. Estrogen promotes the growth of about two thirds of breast cancers (those containing estrogen or progesterone receptors and called hormone receptor positive cancers). Because of this, several approaches to blocking the effect of estrogen or lowering estrogen levels are used to treat breast cancer, including selective estrogen receptor modulators (SERMS) and aromatase inhibitors.

Hormone therapy is preferred over chemotherapy for patients with estrogen receptor-positive (ER+) tumors, a disease-free interval of greater than 2 years, or disease that is not life threatening. Tamoxifen is often used first in premenopausal women. Ovarian ablation by surgery, radiation therapy, or use of a luteinizing-releasing hormone agonist (eg, buserelin, goserelin, leuprolide) is a reasonable alternative. Combination therapy of ovarian ablation with tamoxifen therapy is another alternative. If the cancer initially responds to hormone therapy but progresses months or years later, additional forms of hormone therapy may be used sequentially until no further response is seen.

SERMS are a class of compounds that exert various levels of anti-estrogenic activity in the breast and uterus while showing variable estrogenic effects in other tissues. These tissue-specific effects depend upon the level of interaction of the co-activators and co-repressors and other associated proteins with the estrogen receptor. There are currently two major SERMS are currently in use in the clinic and clinical trials; tamoxifen, and raloxifene.

Tamoxifen has been shown to improve survival at all stages of breast cancer, and adjuvant tamoxifen for about 5 years reduces the annual breast cancer death rate by 31% in women with cancers expressing the estrogen receptor. However, the complications of tamoxifen therapy can include hot flushes; vaginal bleeding, discharge; pruritus vulvae; headache; fluid retention; uterine fibroids, endometriosis; endometrial changes including cancer, uterine sarcoma (mostly malignant, mixed Mullerian tumours); cystic ovarian swellings; haematological changes; hypercalcaemia; thromboembolic phenomena; gastrointestinal intolerance; bone, tumour pain; ocular changes; lightheadedness; rash; alopecia; liver enzyme changes; raised triglycerides, pancreatitis; and in rare cases severe hepatic abnormalities and interstitial pneumonitis. Despite approval by the US FDA, only 5-30% of high-risk women agree to take tamoxifen as a preventive agent because of these reported side effects (in particular endometrial cancer, thromboembolic events, and hot flashes).

Raloxifene has been demonstrated to reduce the risk of invasive breast cancer by 44% in women, however in the same study, the risk of fatal stroke was increased by 49%, and complications of raloxifene therapy may include hot flushes; leg cramps; and thromboembolism. Importantly, half of breast cancers are not prevented or delayed by tamoxifen or raloxifene.

Aromatase inhibitors are compounds that inhibit the transformation of androstenedione and testosterone into estrone and estradiol, respectively. There are two classes of aromatase inhibitors, namely steroidal (e.g. exemestane) and nonsteroidal (e.g. anastrazole and letrozole) available. The complications of exemestane therapy can include hot flushes; fatigue; pain including joint pain, musculoskeletal; oedema; gastrointestinal upset; sweating; headache; dizziness; carpal tunnel syndrome; insomnia; depression; rash; alopecia; lymphopenia; thrombocytopenia; and leucopenia. The complications of anastrazole therapy can include hot flushes; asthenia; joint pain, stiffness; vaginal dryness, bleeding; hair thinning; rash; gastrointestinal upset; headache; carpal tunnel syndrome; hypercholesterolaemia; anorexia (mild); somnolence; severe skin reactions; hypersensitivity including anaphylaxis among others. The complications of letrozole therapy can include hot flushes; gastorintestinal upset; fatigue; anorexia; increased appetite, sweating, weight; hypercholesterolaemia; depression; headache; dizziness; alopecia; rash; arthralgia; myalgia; bone pain, fracture; osteoporosis; and peripheral oedema. Aromatase inhibitors are more effective than tamoxifen as first-line therapy for postmenopausal women with advanced breast cancer or as adjuvant therapy in preventing recurrence of breast cancer however, in addition to the possible side effects listed above, the long-term effects of aromatase inhibitors remain to be evaluated.

Fulvestrant, a steroidal ‘pure’ antiestrogen (i.e. it is free of any estrogen-like activity in the absence of estrogens), exerts its action by blocking the binding of estrogens to the estrogen receptor in all tissues—causing generalized estrogen deprivation. The complications of fulvestrant therapy can include hot flushes; nausea; injection site reaction; asthenia; pain; headache; vasodilatation; bone pain; pharyngitis; dyspnoea; raised liver function tests; and less commonly hypersensitivity. While fulvestrant has been shown to be equivalent to tamoxifen as a primary treatment of advanced breast cancer, no difference was observed in median time to progression compared with anastrazole (in patients who had progressed despite prior endocrine therapy).

A significant problem with the anti-estrogen therapies discussed infra is that patients may demonstrate signs of resistance to the drug at first instance, or may develop resistance in the course of therapy. While the cause of anti-estrogen resistance has not been definitively elucidated, one theory is that mutation(s) in the target (i.e. the estrogen receptor or aromatase molecule) result in a lower affinity of the drug for the target.

Ovarian cancer primarily affects peri- and post-menopausal women. Nulliparity, delayed childbearing, and delayed menopause increase risk, as does a personal or family history of endometrial, breast, or colon cancer. Ovarian cancers are histologically diverse, with at least 80% originating in the epithelium, and of these 75% of these cancers are serous cystadenocarcinoma and the rest include mucinous, endometrioid, transitional cell, clear cell, unclassified carcinomas, and Brenner tumor. The remaining 20% of ovarian cancers originate in primary ovarian germ cells or in sex cord and stromal cells or are metastases to the ovary (most commonly, from the breast or gastrointestinal tract). Germ cell cancers usually occur in women <30 and include dysgerminomas, immature teratomas, endodermal sinus tumors, embryonal carcinomas, choriocarcinomas, and polyembryomas. Stromal (sex cord-stromal) cancers include granulosa-theca cell tumors and Sertoli-Leydig cell tumors.

Ovarian cancer spreads by direct extension, exfoliation of cells into the peritoneal cavity (peritoneal seeding), lymphatic dissemination to the pelvis and around the aorta, or, less often, hematogenously to the liver or lungs. Surgery (hysterectomy and bilateral salpingo-oophorectomy (removal of the ovaries and fallopian tupes) is usually indicated. An exception is nonepithelial or low-grade unilateral epithelial cancer in young patients; fertility can be preserved by not removing the unaffected ovary and uterus. All visibly involved tissue is surgically removed if possible.

Following surgery, changes in sex drive are common. Other complications may include hot flashes and other symptoms of menopause, if both ovaries are removed, increased risk of heart disease and osteoporosis; depression and other forms of psychological distress, blood clots in veins of the legs, risk of infection, internal bleeding, and in the case of hysterectomy, urinary incontinence. Radiation therapy is used infrequently. Chemotherapy may involve topotecan, liposomal doxorubicin, docetaxel, vinorelbine, gemcitabine, hexamethylmelamine, and oral etoposide, and bleomycin.

The complications of topotecan therapy may include haematological and CNS disturbances; fever; infection, sepsis including fatalities; gastrointestinal upset; fatigue; asthenia; alopecia; anorexia; increased liver function tests; dyspnoea and cough among others.

The complications of doxorubicin therapy may include myelosuppression; cardiomyopathy, congestive heart failure; gastrointestinal upset; rash; opportunistic infections; palmar plantar erythrodysaesthesia; severe skin, infusion reactions; extravasation injury; alopecia; myalgia and neuropathy among others.

The complications of vinorelbine therapy may include haematological toxicity; neurological disturbances; gastrointestinal upset; fatigue, fever, arthralgia, myalgia; ischaemic cardiac disease; respiratory distress especially with concomitant mitomycin; and alopecia.

The complications of etoposide therapy may include myelosuppression; gastrointestinal upset; alopecia; and hypotension among others.

The complications of bleomycin therapy may include pulmonary, mucocutaneous toxicity; dermatological changes; renal and hepatic toxicity; hypersensitivity reactions; fever; chills; headache; tiredness; GI upset and anorexia among others.

Cancer of the endometrium is another gynecological cancer that causes significant morbidity and mortality. Endometrial cancer refers to several types of malignancy which arise from the endometrium, or lining of the uterus. Endometrial cancers are the most common gynecologic cancers in the United States, with over 35,000 women diagnosed each year in the U.S. The most common subtype, endometrioid adenocarcinoma, typically occurs within a few decades of menopause, is associated with excessive estrogen exposure, often develops in the setting of endometrial hyperplasia, and presents most often with vaginal bleeding. Because symptoms usually bring the disease to medical attention early in its course, endometrial cancer is only the third most common cause of gynecologic cancer death (behind ovarian and cervical cancer).

Endometrial cancer may sometimes be referred to as uterine cancer. However, different cancers may develop from other tissues of the uterus, including cervical cancer, sarcoma of the myometrium, and trophoblastic disease.

The primary treatment is surgical, typically involving abdominal hysterectomy, and removal of both ovaries and any suspicious pelvic and para-aortic lymph nodes,

Women who are at increased risk for recurrence are often offered surgery in combination with radiation therapy. Chemotherapy may also be considered in some cases such as cisplatin, carboplatin, doxorubicin, and paclitaxel. The side effects of Doxorubicin and Paclitaxel have been considered supra, while those for cisplatin and carboplating include nephrotoxicity, ototoxicity, vestibular toxicity, myelosuppression, anemia, nausea and vomiting, diarrhea, neurotoxicity, muscle cramps, ocular toxicity, anaphylactic-like reactions, and hepatotoxicity,

Thus, the prior art describes many treatment modalities that either physically remove or destroy cells involved in gynecological cancers. Other approaches concentrate on blocking the estrogen receptor by chemical means and by inhibition of the production of estrone and estradiol. From the foregoing description of the prior art, it is clear that every treatment has at least one problem, and may therefore be unsuitable for certain classes of patient. It is an aspect of the present invention to overcome or alleviate a problem of the prior art by providing alternative treatments for breast cancer.

A reference herein to a patent document or other matter which is given as prior art is not to be taken as an admission that that document or matter was, known or that the information it contains was part of the common general knowledge as at the priority date of any of the claims.

Throughout the description and claims of the specification, the word “comprise” and variations of the word, such as “comprising” and “comprises”, is not intended to exclude other additives, components, integers or steps.

### SUMMARY OF THE INVENTION

In one aspect, the present invention provides a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased. The level of biologically available estrogen or androgen may be measured in the blood of the subject. The level of biologically available estrogen may also be measured in a breast cell or an ovarian cell of the subject, or the level of biologically available androgen is measured in an endometrial cell of the subject.

In one form of the invention the polypeptide is such that upon administration of the polypeptide the level of biologically available estrogen or androgen is decreased such that the growth of a breast cancer cell, an ovarian cancer cell or an endometrial cancer cell in the subject is decreased or substantially arrested.

In one embodiment, the polypeptide has an affinity or avidity for an estrogen or androgen that is equal to or greater than the affinity or avidity between the estrogen or the androgen and a protein that naturally binds to the estrogen or the androgen.

In another embodiment, the polypeptide has an affinity or avidity for estradiol or testosterone that is equal to or greater than the affinity or avidity between estradiol and sex hormone binding globulin, or testosterone and sex hormone binding globulin.

In a further embodiment the polypeptide has an affinity or avidity for estradiol or testosterone that is equal to or greater than the affinity or avidity between estradiol and the estrogen receptor, or testosterone and the androgen receptor.

In one form of the polypeptide the estrogen binding region comprises the estrogen binding domain from the human estrogen receptor, or a functional equivalent thereof, or the androgen binding region comprises the androgen binding domain from the human androgen receptor, or a functional equivalent thereof. The estrogen or androgen binding region may also comprise the estrogen or androgen binding domain from sex hormone binding globulin, or a functional equivalent thereof.

In one embodiment, the polypeptide has a single estrogen or androgen binding region. In another embodiment, the polypeptide may comprise a carrier region such as the Fc region of human IgG.

In one form of the polypeptide, the polypeptide is capable of entering a breast cell, an ovarian cell, or an endometrial cell.

The polypeptide may be in the form of a fusion protein, a monoclonal antibody, a polyclonal antibody, or a single chain antibody. The polypeptide may also comprise a multimerisation domain.

In another aspect the present invention provides a nucleic acid molecule capable of encoding a polypeptide as described herein, and also a vector comprising that nucleic acid.

In a further aspect the present invention provides a composition comprising a polypeptide as described herein and a pharmaceutically acceptable carrier.

In yet a further aspect the present invention provides a method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand. The estrogen-related cancer may be breast cancer or ovarian cancer, while the androgen-related cancer may be endometrial cancer. In one form of the method, the ligand is a polypeptide as described herein.

In one embodiment of the method the level of biologically available estrogen is measured in a breast cell or an ovarian cell. In another embodiment the level of biologically available androgen is measured in an endometrial cell. The level of biologically available estrogen or androgen may be measured in the blood of the subject.

In another aspect the present invention provides a method for treating or preventing an estrogen-related cancer or an androgen-related cancer, the method comprising administering to a subject in need thereof an effective amount of a nucleic acid molecule or a vector as described herein. The estrogen-related cancer may be breast cancer or ovarian cancer, while the androgen-related cancer may be endometrial cancer.

In a further aspect the present invention provides a method for treating or preventing estrogen flare or testosterone flare in the treatment of a subject having estrogen-related cancer with an LHRH agonist or antagonist comprising administering to a subject in need thereof an effective amount of a polypeptide, nucleic acid or vector as described herein.

A further aspect of the present invention provides use of a polypeptide, nucleic acid molecule or vector as described herein in the manufacture of a medicament for the treatment or prevention of an estrogen-related cancer or an androgen-related cancer. The estrogen-related cancer may be breast cancer or ovarian cancer, while the androgen-related cancer may be endometrial cancer.

Yet a further aspect of the present invention provides use of a polypeptide, nucleic acid or vector as described herein in the manufacture of a medicament for the treatment or prevention of estrogen flare or testosterone flare.

### DETAILED DESCRIPTION OF THE INVENTION

In a first aspect the present invention provides a polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased. Anti-estrogen or anti-androgen therapy in the form of a polypeptide capable of binding to and effectively sequestering estrogen or androgen molecules is effective in the treatment of cancers for which estrogen has an involvement (such as breast cancer and ovarian cancer), or where androgen levels are relevant (such as endometrial cancer). Without wishing to be limited by theory, it is thought that sequestration of estrogen or androgen prevents binding of the hormone to its cognate receptor in cancer cells, leading to a positive clinical effect.

This approach is fundamentally distinguished from other chemotherapeutic anti-estrogen modalities that either (i) compete with natural estrogens for the binding site on the estrogen receptor leading to the formation receptor complex that is converted incompletely to the fully activated form (e.g. tamoxifen), or (ii) competitively binding to an enzyme involved in estrogen production in the body (e.g. the aromatase inhibitor anastrazole). Given that the polypeptides of the present invention bind to hormones that have a set chemical structure “escape” variants do not pose any problem. By contrast, prior art therapies target protein molecules, which may mutate leading to a lowered affinity of the drug for the target.

Applicant further proposes that anti-androgen therapy in the form of a polypeptide capable of binding to and effectively sequestering androgen molecules is effective in the treatment of cancers for which androgen has an involvement, such as endometrial cancer. The present invention is distinct from approaches of the prior art that aim to surgically remove the cancer by way of hysterectomy, or the use of mitotic inhibitors such as paclitaxel. It is further proposed that the use of anti-androgen polypeptide may be useful in lowering the levels of estrogen in the blood, given that androgens are precursor molecules in the biosynthesis of estrogens.

Typically, the polypeptide has an affinity or avidity for an estrogen or androgen molecule that is sufficiently high such that upon administration of the polypeptide to a mammalian subject, the polypeptide is capable of decreasing biologically available estrogen or androgen hormone in the blood or a cell of the subject to a level lower than that demonstrated in the subject prior to administration of the polypeptide. As used herein, the term “biologically available estrogen or androgen” means an estrogen or androgen molecule that is capable of exerting its biological activity.

A large proportion of estrogen and androgen in the blood is not biologically available. For example, the majority of estrogen and androgen circulating in the blood is not biologically available, with most (around 97%) bound to serum proteins such sex hormone binding globulin (SHBG) and albumin. Hormone binding to SHBG has an association constant (Ka) of about 1×10⁹ L/mol, while that bound to albumin has a much weaker association with a Ka of about 3×10⁴ L/mol.

As will be understood, the present invention is directed to polypeptides that are capable of decreasing the level of an estrogen or androgen hormone available to bind to its cognate receptor in the subject. For example, in the context of the present invention where the hormone is testosterone, the term “biologically available” means that the testosterone is free for conversion to dihydrotestosterone, which subsequently binds to the androgen receptor. Where the androgen is dihydrotestosterone (typically located intracellularly) the term “biologically available” means that the dihydrotestosterone is free to bind to an androgen receptor. Where the hormone is estradiol, the term “biologically available” means that the hormone is available to bind to the estrogen receptor.

In the context of the present invention, the term “estrogen” is intended to include any naturally occurring steroid compounds involved in the regulation of the estrous cycle, and functioning as the primary female sex hormone. Exemplary estrogens include estrone (3-hydroxy-1,3,5(10)-estratrien-17-one); estradiol (1,3,5(10)-estratriene-3,17beta-diol); and estriol (1,3,5(10)-estratriene-3,16alpha,17beta-triol).

As used herein, the term “androgen” is intended to include any natural occurring steroid compound Androgens involved in the development and maintenance of masculine characteristics in vertebrates by binding to androgen receptors. This includes the activity of the accessory male sex organs and development of male secondary sex characteristics. Exemplary androgens include androstenedione (4-androstene-3,17-dione); 4-hydroxy-androstenedione; 11β-hydroxyandrostenedione (11beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5β-dihydrotestosterone; 5-beta-dihydroxy testosterone (17beta-hydroxy-5beta-androstan-3-one); 11β-hydroxytestosterone (11beta,17beta-dihydroxy-4-androsten-3-one); and 11-ketotestosterone (17beta-hydroxy-4-androsten-3,17-dione).

Estrogens and androgens of the present invention include any functionally equivalent synthetic molecule. Thus, the invention includes polypeptides that bind to hormones that are endogenous, and also those that have been administered to a patient in the course of medical treatment.

In one form of the invention, the level of biologically available estrogen is measured in the blood of the subject, or in a breast or ovarian cell. In another form of the invention the level of biologically available estrogen is decreased such that the growth of a breast cancer cell in the subject is decreased or substantially arrested.

The polypeptide may be of high affinity or low affinity or high avidity or low avidity with respect to estrogen. In one embodiment, the polypeptide has an affinity or avidity for an estrogen that is equal to or greater than the affinity or avidity between the estrogen and a protein that naturally binds to the estrogen. As an example, the polypeptide may have an affinity or avidity for estradiol that is equal to or greater than the affinity or avidity between estradiol and sex hormone binding globulin. In another form of the invention the polypeptide has an affinity or avidity for estradiol that is equal to or greater than for the affinity or avidity between estrogen and the estrogen receptor.

The polypeptide may be of high affinity or low affinity or high avidity or low avidity with respect to androgen. In one embodiment, the polypeptide has an affinity or avidity for an androgen that is equal to or greater than the affinity or avidity between the androgen and a protein that naturally binds to the androgen. As an example, the polypeptide may have an affinity or avidity for testosterone that is equal to or greater than the affinity or avidity between testosterone and sex hormone binding globulin. In another form of the invention the polypeptide has an affinity or avidity for testosterone that is equal to or greater than for the affinity or avidity between testosterone and the androgen receptor.

In one embodiment of the polypeptide the estrogen binding region comprises the estrogen binding domain from the human estrogen receptor, or a functional equivalent thereof. Wurtz et al (J Med. Chem. 1998 May 21; 41(11), the contents of which is herein incorporated by reference) published a three-dimensional model of the human estrogen receptor hormone binding domain. The quality of the model was tested against mutants, which affect the binding properties. A thorough analysis of all published mutants was performed with Insight II to elucidate the effect of the mutations. 45 out of 48 mutants can be explained satisfactorily on the basis of the model. After that, the natural ligand estradiol was docked into the binding pocket to probe its interactions with the protein. Energy minimizations and molecular dynamics calculations were performed for various ligand orientations with Discover 2.7 and the CFF91 force field. The analysis revealed two favorite estradiol orientations in the binding niche of the binding domain forming hydrogen bonds with Arg394, Glu353 and His524. After our analysis, the crystal structure of the ER LBD in complex with estradiol was published (Brzozowski et al. Nature 389, 753-758, 1997, the contents of which is herein incorporated by reference). The amino acid sequence of the human estrogen receptor is as follows:

MTMTLHTKASGMALLHQIQGNELEPLNRPQLKIPLERPLGEVYLDSS
KPAVYNYPEGAAYEFNAAAAANAQVYGQTGLPYGPGSEAAAFGSNGL
GGFPPLNSVSPSPLMLLHPPPQLSPFLQPHGQQVPYYLENEPSGYTV
REAGPPAFYRPNSDNRRQGGRERLASTNDKGSMAMESAKETRYCAVC
NDYASGYHYGVWSCEGCKAFFKRSIQGHNDYMCPATNQCTIDKNRRK
SCQACRLRKCYEVGMMKGGIRKDRRGGRMLKHKRQRDDGEGRGEVGS
AGDMRAANLWPSPLMIKRSKKNSLALSLTADQMVSALLDAEPPILYS
EYDPTRPFSEASMMGLLTNLADRELVHMINWAKRVPGFVDLTLHDQV
HLLECAWLEILMIGLVWRSMEHPGKLLFAPNLLLDRNQGKCVEGMVE
IFDMLLATSSRFRMMNLQGEEFVCLKSIILLNSGVYTFLSSTLKSLE
EKDHIHRVLDKITDTLIHLMAKAGLTLQQQHQRLAQLLLILSHIRHM
SNKGMEHLYSMKCKNVVPLYDLLLEMLDAHRLHAPTSRGGASVEETD
QSHLATAGSTSSHSLQKYYITGEAEGFPATV

In another form of the polypeptide, the androgen binding region comprises the androgen binding domain from the human androgen receptor, or a functional equivalent thereof. The gene encoding the receptor is more than 90 kb long and codes for a protein that has 3 major functional domains. The N-terminal domain, which serves a modulatory function, is encoded by exon 1 (1,586 bp). The DNA-binding domain is encoded by exons 2 and 3 (152 and 117 bp, respectively). The steroid-binding domain is encoded by 5 exons which vary from 131 to 288 by in size. The amino acid sequence of the human androgen receptor protein is described by the following sequence.

MEVQLGLGRVYPRPPSKTYRGAFQNLFQSVREVIQNPGPRHPEAASA
APPGASLLLLQQQQQQQQQQQQQQQQQQQQQETSPRQQQQQQGEDGS
PQAHRRGPTGYLVLDEEQQPSOPQSALECHPERGCVPEPGAAVAASK
GLPQQLPAPPDEDDSAAPSTLSLLGPTFPGLSSCSADLKDILSEAST
MQLLQQQQQEAVSEGSSSGRAREASGAPTSSKDNYLGGTSTISDNAK
ELCKAVSVSMGLGVEALEHLSPGEQLRGDCMYAPLLGVPPAVRPTPC
APLAECKGSLLDDSAGKSTEDTAEYSPFKGGYTKGLEGESLGCSGSA
AAGSSGTLELPSTLSLYKSGALDEAAAYQSRDYYNFPLALAGPPPPP
PPPHPHARIKLENPLDYGSAWAAAAAQCRYGDLASLHGAGAAGPGSG
SPSAAASSSWHTLFTAEEGQLYGPCGGGGGGGGGGGGGGGGGGGGGG
GGEAGAVAPYGYTRPPQGLAGQESDFTAPDVWYPGGMVSRVPYPSPT
CVKSEMGPWMDSYSGPYGDMRLETARDHVLPIDYYFPPQKTCLICGD
EASGCHYGALTCGSCKVFFKRAAEGKQKYLCASRNDCTIDKFRRKNC
PSCRLRKCYEAGMTLGARKLKKLGNLKLQEEGEASSTTSPTEETTQK
LTVSHIEGYECQPIFLNVLEAIEPGVVCAGHDNNQPDSFAALLSSLN
ELGERQLVHVVKWAKALPGFRNLHVDDQMAVIQYSWMGLMVFAMGWR
SFTNVNSRMLYFAPDLVFNEYRMHKSRMYSQCVRMRHLSQEFGWLQI
TPQEFLCMKALLLFSIIPVDGLKNQKFFDELRMNYIKELDRIIACKR
KNPTSCSRRFYQLTKLLDSVQPIARELHQFTFDLLIKSHMVSVDFPE
MMAEIISVQVPKILSGKVKPIYFHTQ

The identity of the steroid binding domain has been the subject of considerable research (Ai et al, Chem Res Toxicol 2003, 16, 1652-1660; Bohl et al, J Biol Chem 2005, 280(45) 37747-37754; Duff and McKewan, Mol Endocrinol 2005, 19(12) 2943-2954; Ong et al, Mol Human Reprod 2002, 8(2) 101-108; Poujol et al, J Biol Chem 2000, 275(31) 24022-24031; Rosa et al, J Clin Endocrinol Metab 87(9) 4378-4382; Marhefka et al, J Med Chem 2001, 44, 1729-1740; Matias et al, J Biol Chem 2000, 275(34) 26164-26171; McDonald et al, Cancer Res 2000, 60, 2317-2322; Sack et al, PNAS 2001, 98(9) 4904-4909; Steketee et al, Int J Cancer 2002, 100, 309-317; the contents of which are all herein incorporated by reference). While the exact residues essential for steroid binding are not known, it is generally accepted that the region spanning the approximately 250 amino acid residues in the C-terminal end of the molecule is involved (Trapman et al (1988). Biochem Biophys Res Commun 153, 241-248, the contents of which is herein incorporated by reference).

In one embodiment of the invention the androgen binding region comprises or consists of the sequence approximately defined by the 230 C-terminal amino acids of the sequence dnnqpd . . . iyfhtq.

Some studies have considered the crystal structure of the steroid binding domain of the human androgen receptor in complex with a synthetic steroid. For example, Sack et al (ibid) propose that the 3-dimensional structure of the receptor includes a typical nuclear receptor ligand binding domain fold. Another study proposes that the steroid binding pocket has been consists of approximately 18 (noncontiguous) amino acid residues that interact with the ligand (Matias et al, ibid). It is emphasized that this study utilized a synthetic steroid ligand (R1881) rather than actual dihydrotestosterone. The binding pocket for dihydrotestosterone may include the same residues as that shown for R1181 or different residues.

Further crystallographic data on the steroid binding domain complexed with agonist predict 11 helices (no helix 2) with two anti-parallel β-sheets arranged in a so-called helical sandwich pattern. In the agonist-bound conformation the carboxy-terminal helix 12 is positioned in an orientation allowing a closure of the steroid binding pocket. The fold of the ligand binding domain upon hormone binding results in a globular structure with an interaction surface for binding of interacting proteins like co-activators.

In one embodiment, the estrogen or androgen binding region comprises or consists of the steroid hormone binding domain of the cognate receptor, but is devoid of regions of the receptor that are not involved in steroid hormone binding.

From the above, it will be understood that the identity of the minimum residues required for binding any given hormone may not have been settled at the filing date of this application. Accordingly, the present invention is not limited to polypeptides comprising any specific region of the receptor. It is therefore to be understood that the scope of the present invention is not necessarily limited to any specific residues as detailed herein.

In any event, the skilled person understands that various alterations may be made to the hormone binding sequence without completely ablating the ability of the sequence to bind estrogen or androgen. Indeed it may be possible to alter the sequence to improve the ability of the domain to bind an estrogen or androgen. Therefore, the scope of the invention extends to functional derivatives of the estrogen binding domain of the estrogen receptor, and to functional equivalents of the androgen binding domain of the androgen receptor. It is expected that certain alterations could be made to the hormone binding domain sequence of the relevant receptor without substantially affecting the ability of the domain to bind hormone. For example, the possibility exists that certain amino acid residues may be deleted, substituted, or repeated. Furthermore, the sequence may be truncated at the C-terminus and/or the N-terminus. Furthermore additional bases may be introduced within the sequence. Indeed, it may be possible to achieve a sequence having an increased affinity or avidity for estrogen or androgen by trialing a number of alterations to the amino acid sequence. The skilled person will be able to ascertain the effect (either positive or negative) on the binding by way of standard association assay with estrogen or androgen, as described herein.

In another form of the polypeptide the androgen or estrogen binding region comprises the estrogen binding domain from the sex hormone binding globulin, or a functional equivalent thereof.

In one form of the invention the steroid hormone binding region of the polypeptide comprises a sequence or sequences derived from the steroid binding domain of the human sex hormone binding protein, or a functional equivalent thereof. The sequence of human SHBG is described by the following sequence:

ESRGPLATSRLLLLLLLLLLRHTRQGWALRPVLPTQSAHDPPAVHLS
NGPGQEPIAVMTFDLTKITKTSSSFEVRTWDPEGVIFYGDTNPKDDW
FMLGLRDGRPEIQLHNHWAQLTVGAGPRLDDGRWHQVEVKMEGDSVL
LEVDGEEVLRLRQVSGPLTSKRHPIMRIALGGLLFPASNLRLPLVPA
LDGCLRRDSWLDKQAEISASAPTSLRSCDVESNPGIFLPPGTQAEFN
LRDIPQPHAEPWAFSLDLGLKQAAGSGHLLALGTPENPSWLSLHLQD
QKVVLSSGSGPGLDLPLVLGLPLQLKLSMSRVVLSQGSKMKALALPP
LGLAPLLNLWAKPQGRLFLGALPGEDSSTSFCLNGLWAQGQRLDVDQ
ALNRSHEIWTHSCPQSPGNGTDASH

The scope of the invention extends to fragments and functional equivalents of the above protein sequence. As discussed supra, SHBG is responsible for binding the vast majority of sex hormones in the serum. Accordingly, in one embodiment of the invention the steroid hormone binding region of the polypeptide includes the steroid binding domain of SHBG, or a functional equivalent thereof. This domain comprises the region defined approximately by amino acid residues 18 to 177.

As discussed supra, the polypeptide is capable of decreasing biologically available estrogen. Exemplary methods for measuring of estrogens, such as estradiol, include both indirect and direct immunoassays, and are discussed in Lee et al. 2006, J Clin Endocrinol Metab. 91(10):3791-7, Blondeau and Robel (1975) Eur. J. Biochem. 55, 375-384, and Mounib et al Journal of Steroid Biochemistry 31: 861-865, 1988) the contents of which are all herein incorporated by reference). Examining estradiol levels within the low postmenopausal range, 0-30 pg/ml (0 to 110 pmol/liter), requires more accurate and sensitive assays than the assay methods typically used to discriminate between postmenopausal and premenopausal levels in the 20- to 30-pg/ml range and were originally developed for use in younger women, with the range of interest exceeding 50 pg/ml (183 pmol/liter). Assays that measure levels of total estrogen in the blood (i.e. free hormone in addition to bound hormone) may not be relevant to an assessment of whether a polypeptide is capable of decreasing biologically available estrogen. A more relevant assay would be one that measures free estrogen. An indicator of free estrogen levels is the free estrogen index (FEI). The FEI may be calculated using total estradiol and SHBG values by the following equation: FEI=estradiol (pg/ml)×0.367/SHBG (nmol/l).

In another form of the invention the polypeptide is capable of decreasing the level of biologically available androgen. Free steroid hormone can also be calculated if total steroid, SHBG, and albumin concentrations are known (Sødergard et al, J Steroid Biochem. 16:801-810; the contents of which is herein incorporated by reference). Methods are also available for determination of free steroid without dialysis. These measurements may be less accurate than those including a dialysis step, especially when the steroid hormone levels are low and SHBG levels are elevated (Rosner W. 1997, J Clin Endocrinol Metabol. 82:2014-2015; the contents of which is herein incorporated by reference; Giraudi et al. 1988. Steroids. 52:423-424; the contents of which is herein incorporated by reference). However, these assays may nevertheless be capable of determining whether or not a polypeptide is capable of decreasing biologically available steroid hormone.

Another method of measuring biologically available androgen is disclosed by Nankin et al 1986 (J Clin Endocrinol Metab. 63:1418-1423; the contents of which is herein incorporated by reference. This method determines the amount of steroid not bound to SHBG and includes that which is nonprotein bound and weakly bound to albumin. The assay method relies on the fact SHBG is precipitated by a lower concentration of ammonium sulfate, 50%, than albumin. Thus by precipitating a serum sample with 50% ammonium sulfate and measuring the steroid value in the supernate, non-SHBG bound or biologically available steroid is measured. This fraction of steroid can also be calculated if total steroid, SHBG, and albumin levels are known.

Further exemplary methods of determining levels of biologically available testosterone are disclosed in de Ronde et al., 2006 (Clin Chem 52(9):1777-1784; the contents of which is herein incorporated by reference). Methods for assaying free dihydrotestosterone (Horst et al Journal of Clinical Endocrinology and Metabolism 45: 522, 1977, the contents of which is herein incorporated by reference), dihydroepiandosterone (Parker and O'Dell Journal of Clinical Endocrinology and Metabolism 47: 600, 1978, the contents of which is herein incorporated by reference).

In determining whether or not a polypeptide is capable of decreasing biologically available estrogen or androgen, the skilled person will understand that it may be necessary to account for the natural variability of estrogen and androgen levels that occur in an individual. It is known that estradiol and testosterone levels fluctuate in an individual according to many factors, including the time of day, the amount of exercise performed, and timing of the estrous cycle. Even in consideration of these variables, by careful planning of sample withdrawal, or by adjusting a measurement obtained from the individual, it will be possible to ascertain whether the level of biologically available estrogen or androgen in an individual (and the resultant effect on the growth of cancer cells) has been affected by the administration of a polypeptide as described herein.

In one form of the invention the polypeptide has an affinity or avidity for estrogen or androgen that is equal to or greater than that noted for natural carriers of estrogen in the body. As discussed supra, natural carriers in the blood include SHBG and serum albumin. It will be appreciated that the binding of estrogen to these natural carriers is reversible, and an equilibrium exists between the bound and unbound form of the hormone. In one form of the invention, to decrease the level of biologically available estradiol or testosterone to below that normally present (for example less than about 3% of total hormone in the blood) the polypeptide has an affinity or avidity for the hormone that is greater than that between the cognate binding protein and the hormone. Thus in one embodiment of the invention, the polypeptide has an association constant for the estrogen or androgen that is greater than that for a natural carrier of estrogen or androgen such as SHBG or albumin.

In one form of the polypeptide, the polypeptide has a single estrogen or androgen binding region. This embodiment of the polypeptide may be advantageous due to the potentially small size of the molecule. A smaller polypeptide may have a longer half life in the circulation, or may elicit a lower level of immune response in the body. A smaller polypeptide may also have a greater ability to enter a cell to neutralize intracellular hormone, such as dihydroxytestosterone.

One form of the invention provides a polypeptide with a carrier region. The role of the carrier region is to perform any one or more of the following functions: to generally improve a pharmacological property of the polypeptide including bioavailability, toxicity, and half life; limit rejection or destruction by an immune response; facilitate the expression or purification of the polypeptide when produced in recombinant form; all as compared with a polypeptide that does not include a carrier region.

In one form of the invention, the carrier region comprises sequence(s) of the Fc region of an IgG molecule. Methods are known in the art for generating Fc-fusion proteins, with a number being available in kit form by companies such as Invivogen (San Diego Calif.). The Invivogen system is based on the pFUSE-Fc range of vectors which include a collection of expression plasmids designed to facilitate the construction of Fc-fusion proteins. The plasmids include wild-type Fc regions from various species and isotypes as they display distinct properties

The plasmids include sequences from human wild type Fc regions of IgG1, IgG2, IgG3 and IgG4. Furthermore, engineered human Fc regions are available that exhibit altered properties.

pFUSE-Fc plasmids feature a backbone with two unique promoters: EF1 prom/HTLV 5′UTR driving the Fc fusion and CMV enh/FerL prom driving the selectable marker Zeocin. The plasmid may also contain an IL2 signal sequence for the generation of Fc-Fusions derived from proteins that are not naturally secreted.

The Fc region binds to the salvage receptor FcRn which protects the fusion protein from lysosomal degradation giving increased half-life in the circulatory system. For example, the serum half-life of a fusion protein including the human IgG3 Fc region is around one week. In another form of the invention the Fc region includes human IgG1, IgG2 or IgG4 sequence which increases the serum half-life to around 3 weeks. Serum half-life and effector functions (if desired) can be modulated by engineering the Fc region to increase or reduce its binding to FcRn, FcγR5 and C1q respectively.

Increasing the serum persistence of a therapeutic antibody is one way to improve efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton P R. et al., 2004. J Biol. Chem. 279(8):6213-6; the contents of which is herein incorporated by reference, Vaccaro C. et al., 2005. Nat. Biotechnol. 23(10):1283-8; the contents of which is herein incorporated by reference).

In one form of the invention, the carrier region comprises sequence(s) of the wild type human Fc IgG1 region, as described by the following sequence, or functional equivalents thereof

THTCPPCPAPELLGGPSVFLFPPKPKDTLMISRTPEVTCVVVDVSHE
DPQVKFNWYVDGVQVHNAKTKPREQQYNSTYRVVSVLTVLHQNWLDG
KEYKCKVSNKALPAPIEKTISKAKGQPREPQVYTLPPSREEMTKNQV
SLTCINKGFYPSDIAVEWESNGQPENNYKTTPPVLDSDGSFFLYSKL
TVDKSRWQQGNVFSCSVMHEALHNHYTQKSLSLSPG

While the polypeptide may be a fusion protein such as that described supra, it will be appreciated that the polypeptide may take any form that is capable of achieving the aim of binding a steroid hormone such that the level of steroid hormone in the blood or a cell is decreased.

In one form of the invention the polypeptide is selected from the group consisting of a fusion protein, a monoclonal antibody, a polyclonal antibody, and a single chain antibody.

For example, the polypeptide may be a therapeutic antibody. Many methods are available to the skilled artisan to design therapeutic antibodies that are capable of binding to a predetermined target, persist in the circulation for a sufficient period of time, and cause minimal adverse reaction on the part of the host (Carter, Nature Reviews (Immunology) Volume 6, 2006; the contents of which is herein incorporated by reference).

In one embodiment, the therapeutic antibody is a single clone of a specific antibody that is produced from a cell line, including a hybridoma cell. There are four classifications of therapeutic antibodies: murine antibodies; chimeric antibodies; humanized antibodies; and fully human antibodies. These different types of antibodies are distinguishable by the percentage of mouse to human parts making up the antibodies. A murine antibody contains 100% mouse sequence, a chimeric antibody contains approximately 30% mouse sequence, and humanized and fully human antibodies contain only 5-10% mouse residues.

Fully murine antibodies have been approved for human use on transplant rejection and colorectal cancer. However, these antibodies are seen by the human immune system as foreign and may need further engineering to be acceptable as a therapeutic.

Chimeric antibodies are a genetically engineered fusion of parts of a mouse antibody with parts of a human antibody. Generally, chimeric antibodies contain approximately 33% mouse protein and 67% human protein. They combine the specificity of the murine antibody with the efficient human immune system interaction of a human antibody. Chimeric antibodies can trigger an immune response and may require further engineering before use as a therapeutic. In one form of the invention, the polypeptides include approximately 67% human protein sequences.

Humanized antibodies are genetically engineered such that the minimum mouse part from a murine antibody is transplanted onto a human antibody. Typically, humanized antibodies are 5-10% mouse and 90-95% human. Humanized antibodies counter adverse immune responses seen in murine and chimeric antibodies. Data from marketed humanized antibodies and those in clinical trials show that humanized antibodies exhibit minimal or no response of the human immune system against them. Examples of humanized antibodies include Enbrel and Remicade®. In one form of the invention, the polypeptides are based on the non-ligand specific sequences included in the Enbrel® or Remicade antibodies.

Fully human antibodies are derived from transgenic mice carrying human antibody genes or from human cells. An example of this is the Humira® antibody. In one form of the invention, the polypeptide of the present invention is based on the non-ligand specific sequences included in the Humira® antibody.

The polypeptide may be a single chain antibody (scFv), which is an engineered antibody derivative that includes heavy- and lightchain variable regions joined by a peptide linker. ScFv antibody fragments are potentially more effective than unmodified IgG antibodies. The reduced size of 27-30 kDa allows penetration of tissues and solid tumors more readily (Huston et al. (1993). Int. Rev. Immunol. 10, 195-217; the contents of which is herein incorporated by reference). Methods are known in the art for producing and screening scFv libraries for activity, with exemplary methods being disclosed in is disclosed by Walter et at 2001, Comb Chem High Throughput Screen; 4(2)193-205; the contents of which is herein incorporated by reference.

The polypeptide may have greater efficacy as a therapeutic if in the form of a multimer. The polypeptide may be effective, or have improved efficacy when present as a homodimer, homotrimer, or homotetramer; or as a heterodimer, heterotrimer, or heterotetramer. In these cases, the polypeptide may require multimerisation sequences to facilitate the correct association of the monomeric units. Thus, in one embodiment the polypeptide comprises a multimerisation region. It is anticipated that where the steroid binding region of the polypeptide comprises sequences from SHBG, a multimerisation region may be included.

The present invention also provides a nucleic acid molecule capable of encoding a polypeptide as described herein, and a vector comprising a nucleic acid molecule as described herein. These nucleic acid molecules and vectors will be useful in methods for the recombinant production of the subject polypeptides as well as gene therapy methods for the treatment or prevention of cancer.

Further provided is a composition comprising a polypeptide as described herein and a pharmaceutically acceptable carrier. The skilled person will be enabled to select the appropriate carrier(s) to include in the composition. Potentially suitable carriers include a diluent, adjuvant, excipient, or vehicle with which the polypeptide is administered. Diluents include sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin.

The polypeptides of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Furthermore, aqueous compositions useful for practicing the methods of the invention have physiologically compatible pH and osmolality. One or more physiologically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers are included in an amount required to maintain pH of the composition in a physiologically acceptable range. One or more physiologically acceptable salts can be included in the composition in an amount sufficient to bring osmolality of the composition into an acceptable range. Such salts include those having sodium, potassium or ammonium cations and chloride, citrate, ascorbate, borate, phosphate, bicarbonate, sulfate, thiosulfate or bisulfite anions.

In another aspect the present invention provides a method for treating or preventing an estrogen-related cancer or an androgen-related cancer in a subject, the method comprising administering to a subject in need thereof an effective amount of a ligand capable of binding estrogen or androgen in the subject, such that the level of biologically available estrogen or androgen in the subject is decreased as compared with the level of biologically available estrogen or androgen present in the subject prior to administration of the ligand.

As used herein, the term “estrogen-related cancer” is intended to include any cancer that includes a cell that demonstrates estrogen sensitive growth, proliferation or differentiation. In one form of the method, the estrogen-related cancer is selected from the group consisting of breast cancer and ovarian cancer.

As used herein, the term “androgen-related cancer” is intended to include any cancer that includes a cell that demonstrates androgen sensitive growth, proliferation or differentiation. In one form of the method, the androgen-related cancer is endometrial cancer.

As discussed supra in describing properties of the polypeptides, the level of biologically available hormone may be measured in the blood of the subject. Alternatively, the level of biologically available estrogen may be measured in a breast cell or an ovarian cell. The level of biologically available androgen may be measured in an endometrial cell.

In one form of the method the ligand is a polypeptide as described herein. The amount of the polypeptide that will be effective for its intended therapeutic use can be determined by standard techniques well known to clinicians. Generally, suitable dosage ranges for intravenous administration are generally about 20 to 500 micrograms of active compound per kilogram body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The dosage regime could be arrived at by routine experimentation on the part of the clinician. Generally, the aim of therapy would be to bind all, or the majority of free estrogen or androgen in the blood to the polypeptide. In deciding an effective dose, the amount of polypeptide could be titrated from a low level up to a level whereby the level of biologically available hormone is undetectable. Methods of assaying biologically available estrogens and androgens are known in the art, as discussed elsewhere herein. Alternatively, it may be possible to theoretically estimate (for example on a molar basis) the amount of polypeptide required to neutralize substantially all free hormone. Alternatively, the amount could be ascertained empirically by performing a trial comparing the dosage with clinical effect. This may give an indicative mg/kg body weight dosage for successful therapy.

The duration of treatment and regularity of dosage could also be arrived at by theoretical methods, or by reference to the levels of biologically available hormone in the patient and/or clinical effect.

In one form of the method, the level of biologically available steroid hormone is measured in the blood of the subject, and/or in a cell of the subject.

The methods of treatment will be most efficacious where cancer has already been diagnosed. However, it will be appreciated that the polypeptides may be used prophylactically before cancer has been diagnosed. For example, women with a strong family history of breast cancer could have an estradiol-specific polypeptide infused on a regular basis as a preventative measure.

In another aspect the present invention provides a method for treating or preventing an estrogen-related cancer or an androgen-related cancer, the method comprising administering to a subject in need thereof an effective amount of a nucleic acid molecule or a vector according as described herein. Thus, present invention encompasses the use of nucleic acids encoding the polypeptides of the invention for transfection of cells in vitro and in vivo. These nucleic acids can be inserted into any of a number of well-known vectors for transfection of target cells and organisms. The nucleic acids are transfected into cells ex vivo and in vivo, through the interaction of the vector and the target cell. The compositions are administered (e.g., by injection into a muscle) to a subject in an amount sufficient to elicit a therapeutic response. An amount adequate to accomplish this is defined as “an effective amount.”

For gene therapy procedures in the treatment or prevention of human disease, see for example, Van Brunt (1998) Biotechnology 6:1149 1154, the contents of which is incorporated herein by reference. Methods of treatment or prevention including the aforementioned nucleic acid molecules and vectors may include treatment with other compounds useful in the treatment of cancer. The estrogen-related cancer may be selected from the group consisting of breast cancer and ovarian cancer, while the androgen-related cancer may be endometrial cancer.

In a further aspect the present invention provides a method for treating or preventing estrogen flare or testosterone flare in the treatment of a subject having estrogen-related cancer with an LHRH agonist or antagonist comprising administering to a subject in need thereof an effective amount of a polypeptide as described herein. LHRH drugs eventually result in suppression of testosterone and estradiol, however before this occurs production of these hormones actually increases for a period. During the first week of treatment with a LHRH agonist or antagonist, the vastly increased production of testosterone or estradiol may cause the cancer to flare.

Another aspect of the invention provides the use of a polypeptide as described herein in the manufacture of a medicament for the treatment or prevention of an estrogen-related cancer or an androgen-related cancer. The estrogen-related cancer may be selected from the group consisting of breast cancer and ovarian cancer, and the androgen-related cancer may be endometrial cancer.

In a further aspect the present invention provides the use of a polypeptide as described herein in the manufacture of a medicament for the treatment or prevention of estrogen flare or testosterone flare.

The present invention will now be further described by reference to the following non-limiting examples.

### EXAMPLES

### Example 1

### Construction of Estrogen-Binding Polypeptide

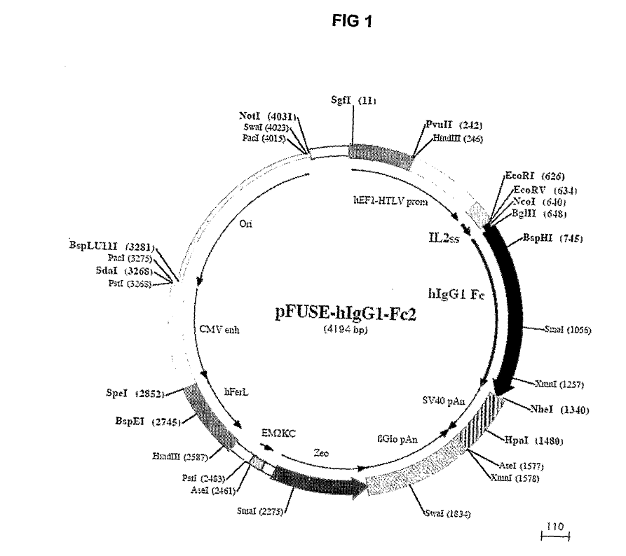

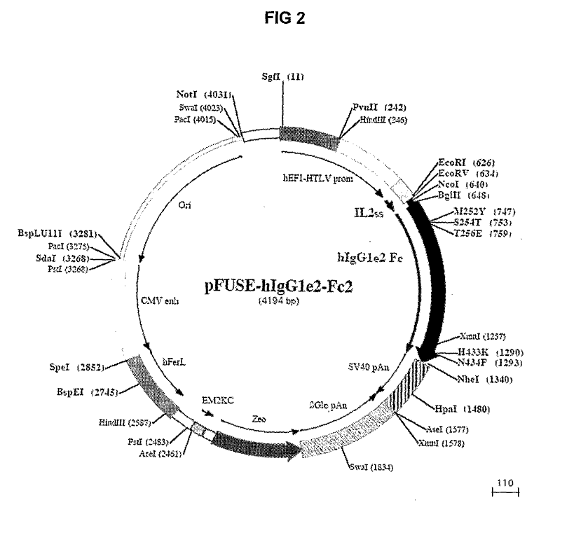

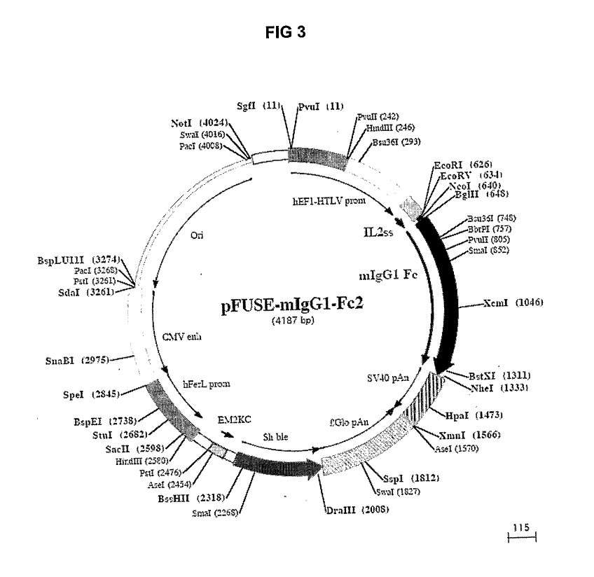

The following coding region for the human estrogen receptor ligand binding domain (723 bp) was subcloned into various vectors (pFUSE-hIgG1-Fc2, pFUSE-hIgG1e2-Fc2, pFUSE-mIgG1-Fc2 from Invivogen) using EcoRI and BgIII RE sites (see FIGS. 1 to 3).

ACCGCCGACC AGATGGTGTC CGCCCTGCTG GACGCCGAGC
CCCCCATCCT GTACAGCGAG TACGACCCCA CCAGGCCCTT
CTCCGAGGCT AGCATGATGG GCCTGCTGAC CAACCTGGCC
GACCGGGAGC TGGTGCACAT GATCAACTGG GCCAAGAGGG
TGCCCGGCTT CGTCGACCTG ACACTGCACG ATCAGGTCCA
CCTGCTGGAA TGCGCCTGGC TGGAAATCCT GATGATCGGC
CTGGTCTGGC GGAGCATGGA ACACCCCGGC AAGCTGCTGT
TCGCCCCCAA CCTGCTGCTG GACAGGAACC AGGGCAAGTG
CGTCGAGGGC ATGGTGGAGA TTTTCGACAT GCTGCTGGCC
ACCTCCAGCA GGTTCAGGAT GATGAACCTG CAGGGCGAGG
AATTTGTGTG CCTGAAGAGC ATCATCCTGC TGAACAGCGG
CGTGTACACC TTCCTGAGCA GCACCCTGAA GAGCCTGGAA
GAGAAGGACC ACATCCACAG GGTGCTGGAC AAGATCACCG
ACACCCTGAT CCACCTGATG GCCAAGGCCG GCCTGACACT
CCAGCAGCAG CACCAGAGGC TGGCCCAGCT GCTGCTGATC
CTGAGCCACA TCAGGCACAT GAGCAACAAG GGGATGGAAC
ACCTGTACAG CATGAAGTGC AAGAACGTGG TGCCCCTGTA
CGATCTGCTC CTGGAAATGC TGGACGCCCA CAGGCTGCAC
GCC

This sequence encodes the 241 C-terminal residues of the human estrogen receptor protein disclosed as follows:

TADQMVSALL DAEPPILYSE YDPTRPFSEA SMMGLLTNLA
DRELVHMINW AKRVPGFVDL TLHDQVHLLE CAWLEILMIG
LVWRSMEHPG KLLFAPNLLL DRNQGKCVEG MVEIFDMLLA
TSSRFRMMNL QGEEFVCLKS IILLNSGVYT FLSSTLKSLE
EKDHIHRVLD KITDTLIHLM AKAGLTLQQQ HQRLAQLLLI
LSHIRHMSNK GMEHLYSMKC KNVVPLYDLL LEMLDAHRLH
A

The various vectors were separately transfected into CHO cells and secreted protein collected. The cell culture supernatant after various times of incubation was spun at 10,000-13,000 rpm for 15 min at 4° C. and concentrated then filtered.

### Cell Line

Mammalian CHO cell cultures were maintained in a Form a Scientific Incubator with 10% carbon dioxide at 37° C. in Dulbecco's Modified Eagle Medium (DMEM) (Gibco). Penicillin (100 U/ml), streptomycin (100 pg/ml) and amphotericin B (25 ng/ml) (Gibco Invitrogen #15240-062) were added to media as standard. As a routine, cells were maintained in the presence of 5% or 10% fetal bovine serum (Gibco Invitrogen #10099-141) unless otherwise stated. Subconfluent cells were passaged with 0.5% trypsin-EDTA (Gibco Invitrogen #15400-054).

### Propagation of DNA Constructs

DNA expression constructs were propagated in supercompetent DH5α E. Coli (Stratagene). To transform bacteria, 1 μg of plasmid DNA was added to 200 μl of bacteria in a microfuge tube and placed on ice for 20 min. Bacteria were then heat shocked at 42° C. for 1.5 min, then replaced on ice for a further 5 min. 1 ml of Luria-Bertani broth (LB) without antibiotics was then added, and the bacteria incubated at 37° C. on a heat block for 1 h. This was then added to 200 ml of LB with penicillin 50 μg/ml and incubated overnight at 37° C. with agitation in a Bioline Shaker (Edwards Instrument Company, Australia). The following morning the bacterial broth were transferred to a large centrifuge tube and spun at 10,000 rpm for 15 min. The supernatant was removed and the pellet dried by inverting the tube on blotting paper. Plasmid DNA was recovered using the Wizard® Plus Midipreps DNA purification system (Promega #A7640). The pellet was resuspended in 3 ml of Cell Resuspension Solution (50 mM Tris-HCl pH 7.5, 10 mM EDTA, 100 pg/ml RNase A) and an equal volume of Cell Lysis Solution added (0.2 M NaOH, 1% SDS). This was mixed by inversion four times. 3 ml of neutralization solution (1.32 M potassium acetate pH 4.8) was then added, and the solution again mixed by inversion. This was centrifuged at 14,000 g for 15 min at 4° C. The supernatant was then carefully decanted to a new tube by straining through muslin cloth. 10 ml of resuspended DNA purification resin was added to the DNA solution and mixed thoroughly. The Midi column tip was inserted into a vacuum pump, the DNA solution/resin mixture added to the column, and the vacuum applied. Once the solution was passed through the column it was washed twice by adding 15 ml of Column Wash Solution and applying the vacuum until the solution had drawn through. After the last wash the column was sharply incised to isolate the column reservoir which was transferred to a microfuge tube and spun at 13,000 rpm for 2 min to remove any residual wash solution. 100 μl of pre-heated nuclease-free water was added and the DNA eluted by centrifuging at 13,000 rpm for 20 sec in a fresh tube. DNA concentration was measured by absorbance spectroscopy (Perkin Elmer MBA2000).

### Examination of DNA Products by Gel Electrophoresis

The DNA products of polymerase chain reactions or restriction enzyme digests of plasmid DNA were analysed by agarose gel electrophoresis. Agarose (1-1.2%) was dissolved in TAE buffer (40 mM Tris acetate, 2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol was added to the samples before electrophoresis. Electrophoresis was conducted at approximately 100V in 1×TAE. DNA samples were visualized under ultraviolet light (254 nm).

### Polypeptide Fusion Protein Transfection and Expression in CHO Cells

Plasmids encoding polypeptide fusion proteins were transfected into CHO cells using Fugene HD (Roche, Cat N^{o}: 04709691001) and selected with Zeocin (Invitrogen, Cat N^{o}:R250-01). 2-5×10⁶ cells were then grown in 100-250 ml CHO-S-SFM II (Invitrogen, Cat N^{o}: 12052-062) for 4-7 days. The cell culture was spun and the supernatant concentrated (using Amicon Ultra 15-50 kDa concentrators, Millipore Cat N^{o}: UFC905024).

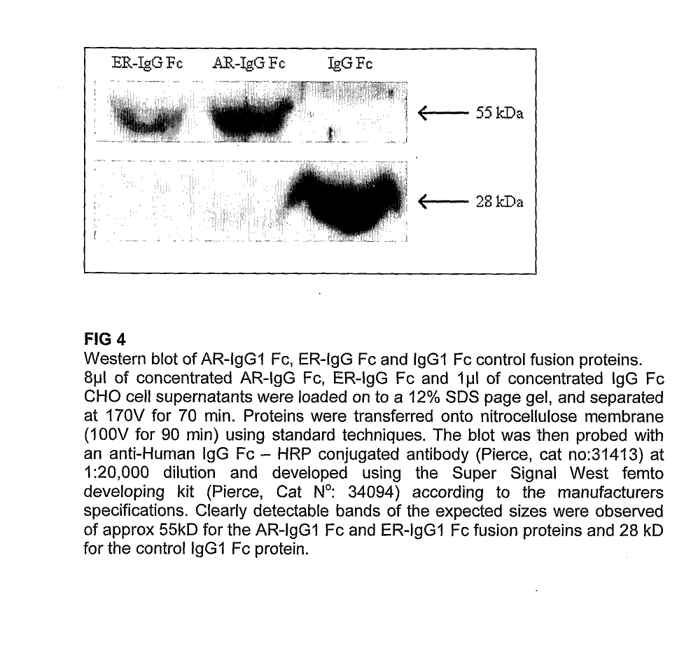

8 μl of concentrated ER-IgG Fc and 1 μl of concentrated IgG Fc supernatant were loaded on to a 12% SDS page gel and run at 170V for 70 min. The gel was then transferred on to nitrocellulose membrane (100V for 90 min) using standard protocols. The membrane was then probed with Anti-Hu IgG Fc-HRP antibody (Pierce, 31413) conjugated at 1:20,000 and developed using the super signal west femto developing kit (Pierce, Cat N^{o}: 34094) according to the manufacturers specifications. Results are as depicted in FIG. 4. Clear expression of a single predominant polypeptide of size approx 55 kD was observed for both the ER-IgG1 Fc fusion protein as well as the AR-IgG1 Fc fusion protein. The control IgG1 Fc control protein of the correct size (28 kD) was also clearly apparent (FIG. 4).

### Example 2

### Construction of Androgen-Binding Polypeptide

The following coding region for human androgen receptor ligand binding domain (690 bp) were subcloned into various vectors (pFUSE-hIgG1-Fc2, pFUSE-hIgG1e2-Fc2, pFUSE-mIgG1-Fc2 from Invivogen) using EcoRI and BgIII RE sites (see FIGS. 1 to 3).

GACAACAACCAGCCCGACAGCTTCGCCGCCCTGCTGTCCAGCCTGAAC
GAGCTGGGCGAGAGGCAGCTGGTGCACGTGGTGAAGTGGGCCAAGGCC
CTGCCCGGCTTCAGAAACCTGCACGTGGACGACCAGATGGCCGTGATC
CAGTACAGCTGGATGGGCCTGATGGTGTTCGCTATGGGCTGGCGGAGC
TTCACCAACGTGAACAGCAGGATGCTGTACTTCGCCCCCGACCTGGTG
TTCAACGAGTACAGGATGCACAAGAGCAGGATGTACAGCCAGTGCGTG
AGGATGAGGCACCTGAGCCAGGAATTTGGCTGGCTGCAGATCACCCCC
CAGGAATTTCTGTGCATGAAGGCCCTGCTGCTGTTCAGCATCATCCCC
GTGGACGGCCTGAAGAACCAGAAGTTCTTCGACGAGCTGCGGATGAAC
TACATCAAAGAGCTGGACAGGATCATCGCCTGCAAGAGGAAGAACCCC
ACCTCCTGCAGCAGAAGGTTCTACCAGCTGACCAAGCTGCTGGACAGC
GTGCAGCCCATCGCCAGAGAGCTGCACCAGTTCACCTTCGACCTGCTG
ATCAAGAGCCACATGGTGTCCGTGGACTTCCCCGAGATGATGGCCGAG
ATCATCAGCGTGCAGGTGCCCAAGATCCTGAGCGGCAAGGTCAAGCCC
ATCTACTTCCACACCCAG

This sequence encodes the 230 C-terminal residues of the human androgen receptor protein.

The various vectors were separately transfected into CHO cells and secreted protein collected. The cell culture supernatant after various times of incubation was spun at 10,000-13,000 rpm for 15 min at 4° C. and concentrated then filtered.

### Cell Line

Mammalian CHO cell cultures were maintained in a Form a Scientific Incubator with 10% carbon dioxide at 37° C. in Dulbecco's Modified Eagle Medium (DMEM) (Gibco). Penicillin (100 U/ml), streptomycin (100 μg/ml) and amphotericin B (25 ng/ml) (Gibco Invitrogen #15240-062) were added to media as standard. As a routine, cells were maintained in the presence of 5% or 10% fetal bovine serum (Gibco Invitrogen #10099-141) unless otherwise stated. Subconfluent cells were passaged with 0.5% trypsin-EDTA (Gibco Invitrogen #15400-054).

### Propagation of DNA Constructs

DNA expression constructs were propagated in supercompetent DH5α E. Coli (Stratagene). To transform bacteria, 1 μg of plasmid DNA was added to 200 μl of bacteria in a microfuge tube and placed on ice for 20 min. Bacteria were then heat shocked at 42° C. for 1.5 min, then replaced on ice for a further 5 min. 1 ml of Luria-Bertani broth (LB) without antibiotics was then added, and the bacteria incubated at 37° C. on a heat block for 1 h. This was then added to 200 ml of LB with penicillin 50 μg/ml and incubated overnight at 37° C. with agitation in a Bioline Shaker (Edwards Instrument Company, Australia). The following morning the bacterial broth were transferred to a large centrifuge tube and spun at 10,000 rpm for 15 min. The supernatant was removed and the pellet dried by inverting the tube on blotting paper. Plasmid DNA was recovered using the Wizard® Plus Midipreps DNA purification system (Promega #A7640). The pellet was resuspended in 3 ml of Cell Resuspension Solution (50 mM Tris-HCl pH 7.5, 10 mM EDTA, 100 μg/ml RNase A) and an equal volume of Cell Lysis Solution added (0.2 M NaOH, 1% SDS). This was mixed by inversion four times. 3 ml of neutralization solution (1.32 M potassium acetate pH 4.8) was then added, and the solution again mixed by inversion. This was centrifuged at 14,000 g for 15 min at 4° C. The supernatant was then carefully decanted to a new tube by straining through muslin cloth. 10 ml of resuspended DNA purification resin was added to the DNA solution and mixed thoroughly. The Midi column tip was inserted into a vacuum pump, the DNA solution/resin mixture added to the column, and the vacuum applied. Once the solution was passed through the column it was washed twice by adding 15 ml of Column Wash Solution and applying the vacuum until the solution had drawn through. After the last wash the column was sharply incised to isolate the column reservoir which was transferred to a microfuge tube and spun at 13,000 rpm for 2 min to remove any residual wash solution. 100 μl of pre-heated nuclease-free water was added and the DNA eluted by centrifuging at 13,000 rpm for 20 sec in a fresh tube. DNA concentration was measured by absorbance spectroscopy (Perkin Elmer MBA2000).

### Examination of DNA Products by Gel Electrophoresis

The DNA products of polymerase chain reactions or restriction enzyme digests of plasmid DNA were analysed by agarose gel electrophoresis. Agarose (1-1.2%) was dissolved in TAE buffer (40 mM Tris acetate, 0.2 mM EDTA pH 8.5) containing 0.5 μg/ml ethidium bromide. A DNA loading dye consisting of 0.2% w/v xylene cyanol, 0.2% bromophenol blue, 40 mM Tris acetate, 2 mM EDTA pH 8.5 and 50% glycerol was added to the samples before electrophoresis. Electrophoresis was conducted at approximately 100V in 1×TAE. DNA samples were visualized under ultraviolet light (254 nm).

### Polypeptide Fusion Protein Transfection and Expression in CHO Cells

The pFUSE-AR-hIgG1e2-Fc2 plasmid encoding the AR-LBD-IgG1FC polypeptide fusion protein was transfected into CHO cells (ATCC) using Fugene HD (Roche, Cat N^{o}: 04709691001) and selected with Zeocin (Invitrogen, Cat N^{o}:R250-01). 2-5×10⁶ cells were then grown in 100-250 ml CHO-S-SFM II serum free suspension medium (Invitrogen, Cat N^{o}: 12052-062) for 4-7 days. The cell culture was spun and the supernatant concentrated (using Amicon Ultra 15-50 kDa concentrators, Millipore Cat N^{o}: UFC905024).

### Analysis of Fusion Protein Expression Levels

8 μl of concentrated AR or ER-LBD IgG Fc supernatant concentrates and 1 μl of concentrated IgG Fc control supernatants were loaded on to a 12% SDS page gel, and run at 170V for 70 min. The electrophoresed proteins were transferred on to a nitrocellulose membrane (100V for 90 min) using standard techniques. The nitrocellulose membranes were then probed with an Anti-Hu IgG Fc-HRP conjugate (Pierce, cat no: 31413) at 1:20,000 dilution and developed using the Super Signal West Femto developing kit (Pierce, Cat N^{o}: 34094) according to the manufacturers specifications. The results are depicted in FIG. 4.

Clear expression of a single predominant polypeptide of size approx 55 kD was observed for both a AR-IgG1 Fc fusion protein as well as the ER-IgG1 Fc fusion protein. The control IgG1 Fc control protein of the correct size (28 kD) was also clearly apparent (FIG. 4).

### Example 3

### Efficacy of Estrogen-Binding Polypeptide by In Vitro Assay

A human hormone sensitive breast cancer cell line, MCF-7, is exposed to the ° ER-LBD-IgG1FC fusion protein as described in Example 1. The effects of the polypeptide on the growth and proliferation of the cells is then assessed.

As a control for hormone ablation therapy, the cells are cultured in hormone depleted serum (Charcoal stripped serum, CSS) as well as in normal serum to demonstrate growth in normal levels of estrogen.

Cell Culture. Human breast adenocarcinoma (MCF-7) cell line (ATCC, USA) is routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells are maintained at 37° C. in 5% CO₂. Estrogen is purchased from Sigma-Fluka (St Louis, Mo., USA) and dissolved in 100% ethanol, then further diluted to make 100 μM working stock solutions in phenol-red RPMI 1640 (Invitrogen, Auckland, New Zealand) and serial dilutions are made in 5% charcoal strip serum (CSS, Hyclone #SH30068.03) for in vitro experiments.

In Vitro—proliferation assay. 4×10³ MCF-7 cells are plated per well in a Falcon 96-well plate in growth media either with 5% FBS or 5% CSS with/or without estrogen (0.001, 0.01, 0.1, 1.0 and 10.0 μM) and cultured over 14 days at 5% CO₂/37° C. Cells are trypsinised and counted with trypan blue after 7 days in culture, then cells are reseeded at the same density as above into another 96-well plate with the above growth media for another 7 days in culture. At day 14, cells are washed once with PBS and labelled with calcein (C1430, Molecular Probes, Oregon, USA) at 1 mM final concentration in PBS. Calcein positive cells are detected by using an FLUOstar OPTIMA plate reader (BMG Labtech, Victoria, Australia).

Similarly, to see the effect of estrogen-binding peptide on human oestrogen dependent MCF-7 cells: 4×10³ MCF-7 cells were seeded as above in a 96-well plate cultured in growth media containing 5% CSS with estrogen (0.001, 0.01, 0.1, 1.0 and 10.0 μM μM). Cells were treated with either ER-LBD IgG1Fc fusion protein (20, 50, 100 ng/ml) or IgG1Fc control protein (20, 50, 100 ng/ml). Experiments were performed in 4 replicates per treatment group.

### Example 4

### Efficacy of Androgen-Binding Polypeptide by In Vitro Assay

A human hormone sensitive prostate cancer cell line, LNCaP, was exposed to the AR-LBD-IgG1FC fusion protein as described in Example 2. The effects of the polypeptide on the growth and proliferation of the cells was then assessed.

As a control for hormone ablation therapy, the cells were cultured in hormone depleted serum (Charcoal stripped serum, CSS) as well as in normal serum to demonstrate growth in normal levels of androgens. In addition, LNCaP cells were also cultured in the presence of the non-steroidal antiandrogen nilutamide

### Cell Culture.

The human prostate cancer cell line, LNCaP was obtained from American Type Tissue Collection (ATCC) and was routinely cultured in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were maintained at 37° C. in 5% CO₂.

### In Vitro—Growth Proliferation Study.

2×10³ LNCaP cells were plated per well in a Falcon 96-well plate in 5% CO₂/37° C. in growth medium containing phenol red RPMI 1640 (Invitrogen, Auckland, New Zealand) supplemented with 10% fetal bovine serum (FBS, GIBCO) and 1% antibiotic/antimycotic mixture (Invitrogen, Auckland, New Zealand). Cells were treated with either AR-LBD IgG1Fc fusion protein (12 ng/ml) or IgG1Fc control protein (12 ng/ml). In addition as control, 6 wells were treated with the nonsteroidal antiandrogen nilutamide (0.1 μM) as well as 6 wells with 10% charcoal stripped serum, to simulate steroid free conditions. After 120 hours in culture, cells were washed once with PBS and labelled with calcein (C1430, Molecular Probes, Oregon, USA) at 1 mM final concentration in PBS. Calcein positive cells were detected using a FLUOstar OPTIMA plate reader (BMG Labtech, Victoria, Australia). Experiments were performed in 6 replicates for each treatment condition.

### Statistical Analysis

Data are presented as mean±SEM unless otherwise indicated.

### Results

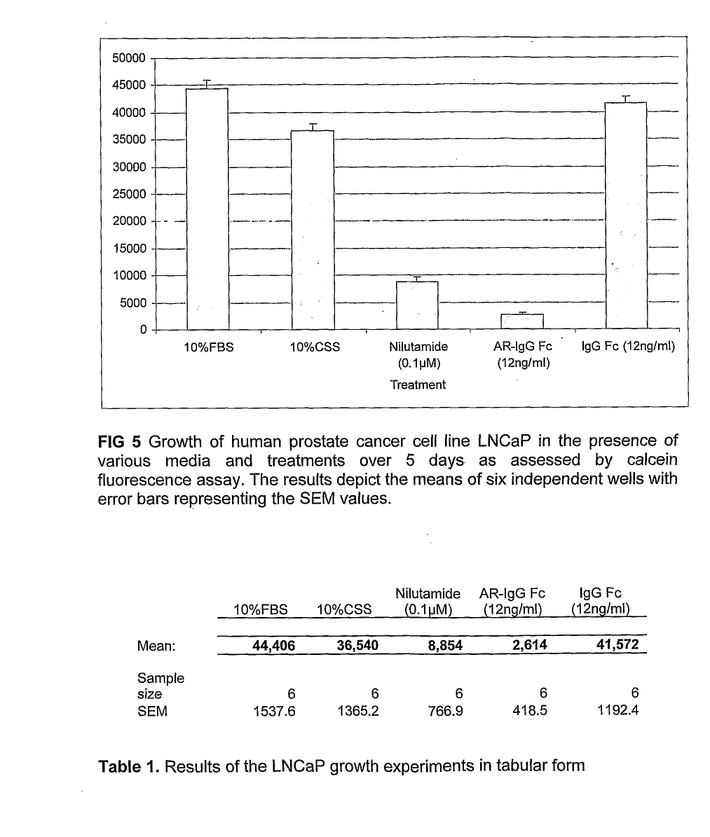

Treatment of the human hormone sensitive prostate cancer LNCaP cells with the AR IgG1 Fc fusion protein produced a dramatic effect on growth after 5 days exposure as assessed by the fluorescent calcein uptake assay. A 94% reduction in viable LNCaP cells was observed in wells treated with the AR IgG1 Fc fusion protein compared to LNCaP cells grown in media with complete 10% serum (FBS) (FIG. 5, Table 1). In comparison, the control IgG1 Fc protein lacking the AR LBD region had only a negligible effect on growth of the LNCaP cells with only a 6% decline in total cell number (FIG. 5, Table 1), indicating that the growth suppression effect is mediated via the androgen binding domain of the fusion protein. Growth of the LNCaP cells in media devoid of steroids, in the charcoal stripped serum (CSS) had only a modest effect on reducing LNCaP cell proliferation in the assay time frame, with a 18% decline observed (FIG. 5, Table 1). Interestingly, the AR IgG1 Fc fusion protein showed superior efficacy to the antiandrogen nilutamide in reducing LNCaP cell proliferation, with nilutamide reducing prostate cancer cell proliferation by 80% (FIG. 5, Table 1).

These results indicate that the AR IgG1 Fc fusion protein is able to suppress androgen mediated growth of prostate cancer cells. However, this suppression is occurring not only via depleting free androgen levels in the exogenous media, as growth of the LNCaP cells in media totally devoid of steroids had only a modest effect on the cellular proliferation. This superior effect of the AR IgG1Fc protein compared to growth in steroid stripped serum indicates that the fusion protein is able to sequester endogenous androgens either internally or externally produced by the LNCaP cells.

### Example 5

### Efficacy of Estrogen-Binding Polypeptide by In Vivo Assay

### Breast Cancer Models

6 week old female balb/c/SCID, mice were housed under sterile conditions in micro-isolators. Antibiotics (Baytril 25) were given via drinking water to all mice.

All mice received a controlled amount of estradiol (up to 30 micrograms per day) that was delivered by subcutaneous hormone pellets. Each group comprised eight mice. One control group had no tumour injected while another was injected with tumour cells but received no treatment.

Orthotopic Breast cancer was established by injection into the mammary fat pad, with 2×10⁶ viable human breast cancer and estrogen receptor positive MCF-7 cells resuspended in 50 μl 10% FCS (Bovogen, Cat N^{o}: SFBS) in RPMI (Invitrogen, Cat N^{o}: 11875) and injected into the right hand mammary fat pad. The injections were carried out in the animal facility under sterile conditions.

### Treatment Arms

Seven days later mice began treatment of weekly intravenous injections with approximately 300 ng of IgG Fc or 300 ng of ER-IgG Fc in 200 μl of CHO-S-SFM II (Invitrogen, Cat N^{o}: 12052-062) via the tail vein.

Pellets for estradiol hormone therapy were implanted either using a stainless steel reusable precision trochar. Each mouse had a small incision and pocket made on the left hand flank with an estradiol pellet deposited (1.7 mg 90 day release pellet, Innovative Research of America, Cat N^{o}: NE-121).

Animals receiving surgery for implantation were administered an anaesthetic of isoflurane. The incision was closed with 4/0 silk.

### Monitoring and Collection of Samples

The end of the experiment was defined as the point when tumours in the untreated control animal groups approach 10% of the animal's normal body weight. This represents a subcutaneous flank tumour diameter of 17 mm in a 25 g mouse. Tumours were monitored and the hair of the SCID mice removed. Mice were euthanised with carbon dioxide, tumours removed, weighed and the dimensions recorded. Specimens were fixed and embedded for future analysis.

Data was Collected and Analysed using Mann-Whitney Test for Significance.

### Error Bars Represent the SEM

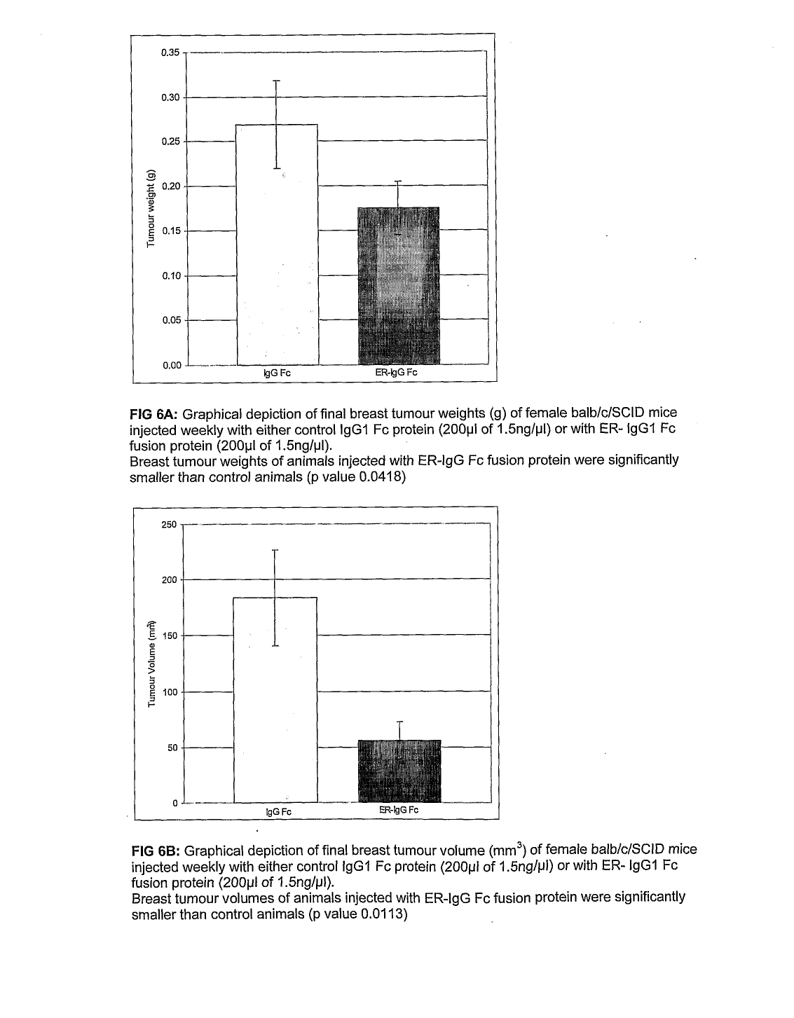

The results are depicted in FIGS. 6A, B. The final tumour weight of the control mice injected with the IgG1 Fc protein averaged 269 mg. However, the final tumour weight of the mice injected with the ER-LBD IgG1 Fc fusion protein was significantly lower at 175 mg (p value 0.0418) (FIG. 6A). There was also a significant effect of the ER-LBD IgG1 Fc fusion protein in inhibiting breast tumour volume throughout the experiment with animals treated with the estrogen binding fusion protein having significantly smaller tumour volumes at the end of the experiment at 56 mm³ (FIG. 6B). This was in marked contrast with animals injected with the control IgG1 protein which developed tumours which were much larger at the end of the experiment at 184 mm³ (p value 0.0113) (FIG. 6B).

### Example 6

### Efficacy of Androgen-Binding Polypeptide by In Vivo Assay

### Rapid Reduction in Circulating Free Testosterone Levels

Athymic balb/c nude male mice, 6 weeks of age, were purchased from the Animal Resources Centre, Perth, Western Australia, and housed in a microisolator. Mice were given free access to standard rodent chow and drinking water throughout all experiments.

5 animals were administered IV tail vein injections of the AR-LBD IgG1Fc fusion protein (25 ng in 2000 of PBS). Three hours after injection the blood of all 5 mice was collected/pooled via mandibular bleeds (approx 100 μL blood per animal) in Lithium/heparin tubes. In addition, 5 control athymic balb/c nude male mice of the same sex and age were similarly bled at the same time and samples pooled. The unclotted blood was then spun at 2500 rpm for 5 min to separate the red blood cells from the serum. 100 μl samples of pooled serum were then run according to the manufacturers specification of the Coat-a-count Free testosterone kit (Siemens, Cat No: TKTF1).

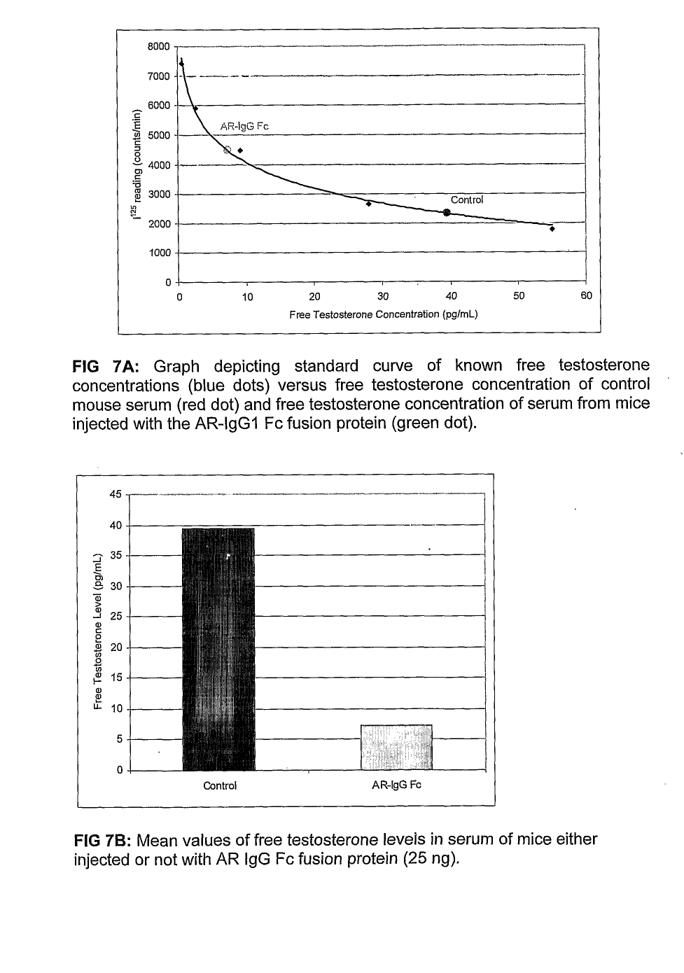

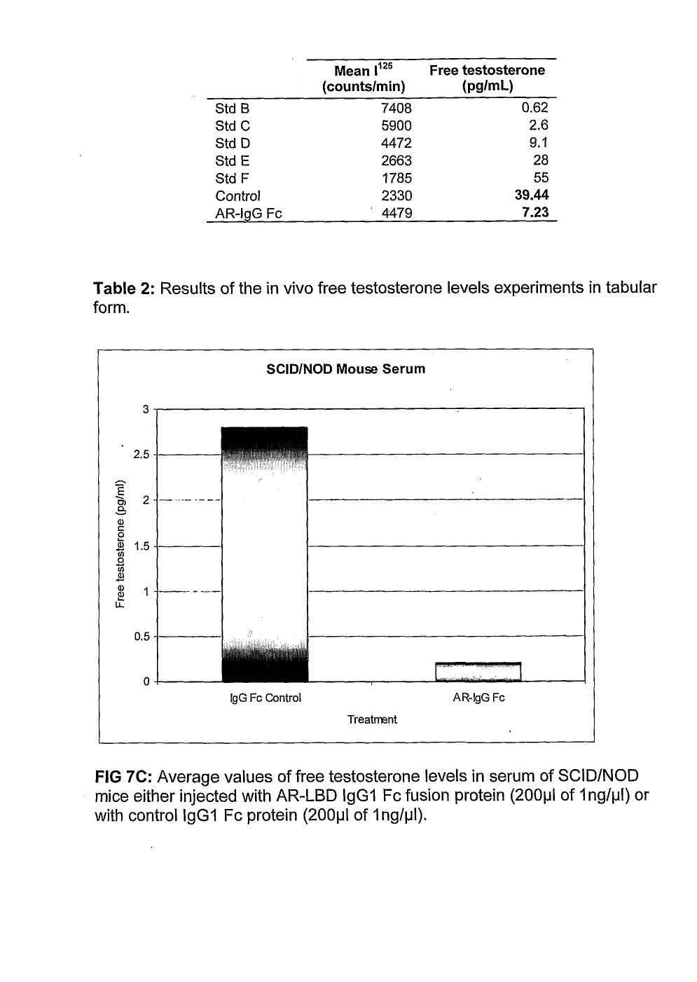

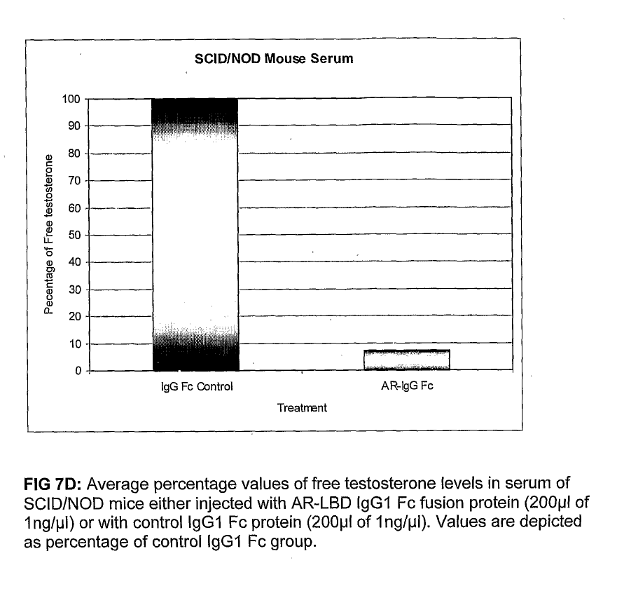

The results are depicted in FIG. 7A, B and Table 2. The free testosterone levels in the serum of the control mice averaged 39.44 pg/ml. However, the free testosterone levels of the mice injected with the AR IgG1 Fc fusion protein was only 7.23 pg/ml. This represents a dramatic 82% decline in bioavailable testosterone levels in only 3 hours after injection.

In a further experiment, 6 SCID/NOD male mice, 5 weeks of age were purchased from the Animal Resources Centre, Perth, Western Australia, and housed in a microisolator. Mice were given free access to standard rodent chow and drinking water throughout all experiments. The animals were then separated into two groups of 3 mice. Three animals in one group were administered IV tail vein injections of the AR-LBD IgG1 Fc fusion protein (200 μl of 1 ng/μl of PBS). Three mice in the other control group, were then administered IV tail vein injections of the control IgG1 Fc protein (200 μl of 1 ng/μl of PBS). Four hours after injection the blood of all 6 mice was collected via mandibular bleeds (approx 100 μl blood per animal) in Lithium/heparin tubes. The unclotted blood was then spun at 2500 rpm for 5 min to separate the red blood cells from the serum. 100 μl samples of pooled serum were then run according to the manufacturers specification of the Coat-a-count Free testosterone kit (Siemens, Cat No: TKTFI).

The results are depicted in FIGS. 7C and D. The free testosterone levels in the serum of the control mice injected with the control IgG1 Fc protein averaged 2.8 pg/ml. However, the free testosterone levels of the mice injected with the AR-LBD IgG1 Fc fusion protein was only 0.2 pg/ml. This represents a dramatic 93% decline in bioavailable testosterone levels only 4 hours after injection.

### Example 7

### Efficacy of Androgen-Binding Polypeptide by In Vivo Assay

A xenograft animal model of an androgen dependent tumor is used to assess efficacy in vivo. 5-7 week old SCID (severe combined immunodeficiency) or athymic balb/c nude male mice were purchased from the Animal Resources Centre, Perth, Western Australia, and housed in microisolators. Mice were given free access to standard rodent chow and drinking water throughout all experiments.

### Subcutaneous Tumour Models

To establish flank prostate tumours, 4×105 washed LNCaP cells were resuspended in 50□l PBS, mixed with an equal volume of Matrigel (BD #354234) and injected subcutaneously into the right flank of 6 week old male nude mice with a 23 G needle. Following tumour cell injection, 100 μl of 1 ng/μl control IgG1 Fc was injected into the flanks of three mice and 100 μl of 1 ng/μl AR-LBD IgG1 Fc fusion protein injected into the flanks of the three remaining mice. Seven days later, a second flank injection of 200 μl of 1 ng/μl IgG1 Fc was administered to the three animals in the control group and 200 μl of 1 ng/μl AR-LBD IgG1 Fc fusion protein was administered to the three animals in the active treatment group. No further treatment was given and the animals were monitored and tumour sizes measured regularly. The experiment was terminated 5 weeks after the initial tumour cell injection, and final tumour volumes and weight were recorded.

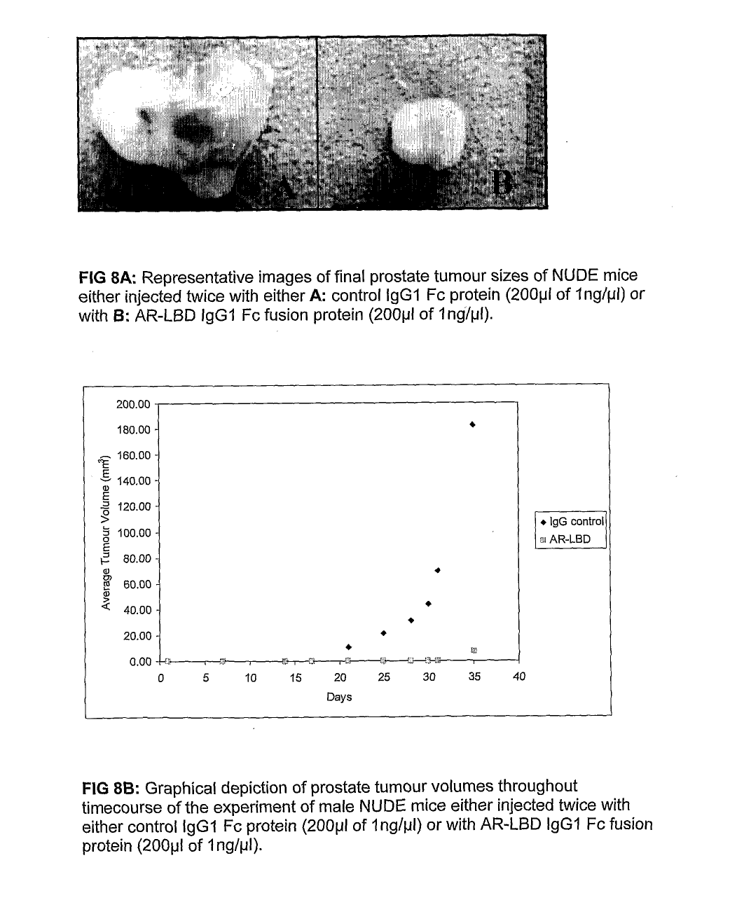

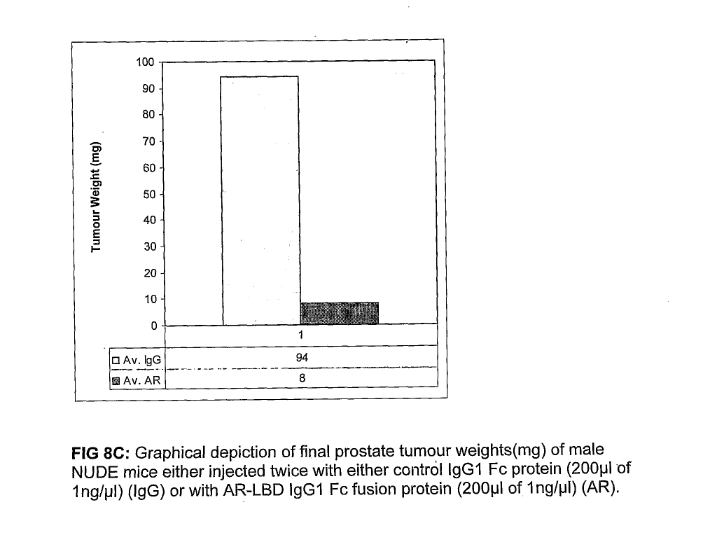

The results are depicted in FIGS. 8A, B and C. The final tumour volume of the control mice injected with the IgG1 Fc protein averaged 182.9 mm3. However, the final tumour volume of the mice injected with the AR-LBD IgG1 Fc fusion protein was only 7.3 mm3 (FIGS. 8A and B). There was also a significant effect of the AR-LBD IgG1 Fc fusion protein in inhibiting prostate tumour growth throughout the experiment with animals treated with the androgen binding fusion protein only developing very small tumours at the end of the experiment (FIG. 7B). This was in marked contrast with animals injected with the control IgG1 protein which developed tumours much earlier and which were much larger at the end of the experiment (FIG. 8B).

There was similarly a very large effect of the AR-LBD IgG1 Fc fusion protein on final tumour weights with average weight being only 8 mg whilst control mice injected with the IgG1 Fc protein averaged 94 mg (FIG. 8C).

### Orthotopic Model of Hormone Dependent Prostate Cancer

Orthotopic tumours are established as follows. Mice (between 6-10 per treatment group) are anaesthetized with a mixture of ketamine 100 mg/kg and xylazine 20 mg/kg injected intraperitoneally to allow a small transverse lower abdominal incision to be made. The bladder, seminal vesicles and prostate are delivered into the wound and 1×10⁶ LNCaP cells in 20 μl of cell culture medium with Matrigel injected into the dorsolateral prostate with a 29 gauge needle. Injections are performed with the aid of an operating microscope at ×10 magnification. A technically satisfactory injection is confirmed by the formation of a subcapsular bleb and the absence of visible leak. The lower urinary tract is replaced and the anterior abdominal wall closed with 4/0 silk. The skin is apposed with surgical staples. Postoperatively the animals are given an intraperitoneal injection of normal saline at a calculated volume of 3-5% of the pre-anaesthetic weight. Mice are recovered under radiant heating lamps until fully mobile.

Animals are divided into treatment groups of 6-10 mice and after different time periods following tumour cell injection are administered IV tail vein injections of the polypepetide at different concentrations (optimised from in vitro experimental results). At the end of the experiment mice are sacrificed by carbon dioxide narcosis. The prostate, seminal vesicles and bladder are removed en bloc, and appendages carefully dissected from the tumour containing prostate if not grossly involved. The tumour containing prostate gland is weighed, and diameter measured in three dimensions with Vernier calipers. The retroperitoneum is explored under magnification cephadally to the level of the renal veins. Lymph nodes found in the para-aortic and para-iliac areas are dissected free and their long axis measured. Tissue for Immunohistochemical staining is embedded in OCT and frozen in liquid nitrogen cooled isopentane. Tumours are stored at −70° C. until analysis.

### Surgical Castration

As controls for hormone ablation therapy, Mice are anaesthetized with a mixture of ketamine 100 mg/kg and xylazine 20 mg/kg injected intraperitoneally to allow a small transverse lower abdominal incision to be made. The lower genitourinary organs are delivered into the wound, the vas deferens and vascular pedicle ligated with 4/0 silk, and the testes excised. The abdomen is closed with 4/0 silk with clips to skin. Mice are recovered on a heating pad until fully recovered.

### Local Tumour Growth in Orthotopic Models of ADPC

At specified times post inoculation (from days 25-42), mice are euthanased by carbon monoxide narcosis and a necroscopy performed. The abdomen is opened in the midline from sternum to pubis and retracted, and the abdominal organs inspected. Under magnification, the urethra is transected at the prostatic apex and the ureters and vas deferentia are identified bilaterally and divided close to the prostate. The specimen is then removed en bloc and the seminal vesicles and bladder dissected free under magnification. The tumour containing prostate gland is then weighed and its dimensions measured in 3 axes with Vernier calipers. Where a discrete nodule is found this is dissected away and weighed separately.

After these measurements, the prostate or tumour is embedded in OCT, snap frozen in liquid nitrogen cooled isopentane and stored at −70° C. until use. Prostate glands without macroscopic tumours are serially sectioned and analysed histologically to confirm the presence of tumour.

Volume of the tumour containing prostate gland is calculated using the formula a*b*c, where a, b and c represent maximum length of the gland measured with Verniers calipers in three dimensions at right angles to one another.

### Example 8

### A Study to Determine the Efficacy and Safety of Estrogen-Specific Polypeptide in Patients with Metastatic Breast Cancer Who have Failed Previous Hormonal Therapy

This study includes up to 15 post-menopausal women with hormone-sensitive (ER+ or PgR+) metastatic breast cancer, who progress on prior hormone therapy. The purpose of this study is to evaluate the safety and efficacy of estrogen-specific polypeptide in patients who progress on prior hormone therapy for breast cancer. Study participants remain on treatment until disease progression or until other treatment discontinuation criteria are met.

This Example is directed to patients who fail primary hormone therapy. While it would be possible (and desirable) to trial the polypeptide in patients with hormone dependent tumours, patients with advanced breast cancer who fail first line hormone therapy are used at first instance for ethical reasons. This approach allows an assessment of whether the polypeptide is well tolerated, and also permits assessment of the effects on levels of biologically available estrogen levels.

### Objectives

The primary objectives of this study are to determine the safety and tolerability of intra venous infusions of the polypeptide binding protein in patients with advanced breast cancer, and to evaluate its pharmacokinetic profile when given as a single IV infusion once every three weeks. Secondary objectives include: to determine whether treatment with polypeptide binding protein can lead to clinical responses; to estimate progression-free survival; to determine whether treatment with polypeptide binding protein can lead to biological responses in patients with advanced breast cancer.

### Study Design

This study describes an open label phase I dose escalation study. After signing informed consent, patients undergo baseline testing to confirm eligibility. Patients then commence treatment with polypeptide binding protein, administered as a single intravenous infusion once every three weeks (one cycle). After four cycles of therapy (12 weeks), patients with stable or responding disease, and who wish to continue on study, are offered treatment extension for up to another four cycles. All patients are assessed for safety 28 days after the last dose of study drug, and where possible, are evaluated three months after their final treatment of study drug. In total, 12-15 patients (4-patients per dose level) are recruited from a variety of multidisciplinary breast-oncology clinics.

### Patient Eligibility

Patients are screened for study eligibility based on the following inclusion and exclusion criteria. To participate in the study a patient should meet the following criteria:
provide written informed consent
be female with histological/cytological confirmation of hormone sensitive breast cancer with evidence of metastatic disease
have one or more measurable lesions

Any of the following is regarded as a criterion for exclusion from the trial:
1. Prior cytotoxic chemotherapy for advanced breast cancer
2. had radiation therapy within 4 weeks prior to provision of consent
3. Treatment with an investigational agent in the last 4 weeks
4. Other co-existing malignancies or malignancies diagnosed within the last 5 years with the exception of non-melanomatous skin cancer
5. Any unresolved chronic toxicity greater than CTC grade 2 from previous anticancer therapy
6. Incomplete healing from previous surgery
7. Absolute neutrophil counts <1×10⁹/l or platelets <100×10⁹/l
8. Serum bilirubin >1.25 times the upper limit of reference range (ULRR)
9. In the opinion of the investigator, any evidence of severe or uncontrolled systemic disease (e.g. unstable or uncompensated respiratory, cardiac, hepatic or renal disease)
10. Serum creatinine >1.5 times the ULRR
11. Alanine aminotransferase (ALT) or aspartate aminotransferase (AST)>2.5 times the ULRR
12. Evidence of any other significant clinical disorder or laboratory finding that makes it undesirable for the patient to participate in the trial
13. Patients may not use unapproved or herbal remedies for breast cancer
14. A history of alcoholism, drug addiction, or any psychiatric condition which in the opinion of the investigator would impair the patient's ability to comply with study procedures.

### Study Agent

The polypeptide is produced in accordance with Example 1. All formulation and packing of the study agent is in accordance with applicable current Good Manufacturing Practice (GMP) for Investigation Medicinal Products as specified by the Therapeutic Goods Administration (Australia) and meet applicable criteria for use in humans.

### Treatment Plan

Three dose levels of polypeptide binding protein are investigated (0.3, 1.0, and 3.0 mg/kg). After enrollment in the 0.3-mg/kg cohort is complete, there is a 2-week waiting period before the 1.0-mg/kg cohort is begun. There is also a 2-week waiting period after the 1.0-mg/kg cohort is enrolled before enrollment of the 3.0-mg/kg cohort is begun.

Individual patient doses are prepared by diluting the appropriate volume of polypeptide binding protein (25 mg/ml) with 0.9% sodium chloride to yield a final concentration of 4 mg/ml. The volume of solution prepared is 25 to 150 ml, depending on the patient's dose and body weight. The polypeptide is infused over a period of no less than 1 hour by a registered nurse or physician's assistant under the guidance of one of the trial investigators. In addition, internists or anesthesiologists are present to oversee the administration of the study agent and aid in the management of adverse events.

All adverse events are graded according to the Common Terminology Criteria for Adverse Events Version 3.0 (Cancer Therapy Evaluation Program, DCTD, NCI, NIH, DHHS, Mar. 31, 2003, http://ctep.cancer.gov). DRT and DLT is based on the first three weeks of treatment. DRT is defined as any Grade 2 non-haematological or Grade 3 haematological toxicity. DLT is defined as any Grade 3/4 non-haematological or Grade 4 haematological toxicity. Patients who require other treatment for progressive breast cancer, such as radiotherapy to new metastatic lesions, surgery or chemotherapy, are removed from the study and are not replaced. Treatment will not be administered if there is Grade 2 haematological and/or non-haematological toxicity. Treatment may be re-initiated once, the toxicity is 5 Grade 1, with treatment delayed for up to two weeks. In the absence of treatment delays, treatment may continue for up to four cycles or until there is disease progression; intercurrent illness prevents further administration of treatment; unacceptable adverse events occur; the patient decides to withdraw from the study; or general or specific changes in the patient's condition render the patients unacceptable for further treatment in the judgment of the trial investigator.

### Pre-Treatment and Treatment Evaluation

At study entry, patients are screened for measurable disease by radionuclide bone scintigraphy and computed tomography of the chest, abdomen and pelvis. In patients with measurable disease, tumour response is assessed according to the Response Evaluation Criteria in Solid Tumours (Therasse, P., et al., J Natl Cancer Inst, 2000. 92(3): p. 205-16). Given the stage of disease at which patients are enrolled, it is anticipated that the majority will have measurable disease at the time of study entry. Toxicity is evaluated according to the Common Terminology Criteria for Adverse Events Version 3.0.

### Sample Collection

Sample collection to determine population pharmacokinetic parameters for polypeptide binding protein is performed in patients accrued to the study. Serial blood samples (10 ml/sample) are collected at the following times: pre-dose (within 60 min prior to study drug administration) and post-dose at 30 min, 1, 2, 4, 6, 24, 48 and 72 h. In addition, trough samples are taken at days 7, 14 and 21, weeks. Blood samples are collected into heparinised vacutainers for assessment of sodium selenate status. The plasma is separated by centrifugation (2000 g at 4° C. for 15 min). Following centrifugation, the plasma is separated into three aliquots (each approximately 1 ml) and placed in identically labelled polypropylene tubes. Samples are frozen at −80° C. until analysis.

### Study Completion

A patient is considered to have completed the study following the evaluations for the primary endpoint after 4 cycles of treatment. However, patients continuing on study and receiving further treatment are followed and data collected. Where possible, all patients are evaluated every three months. The study is closed when the final patient has undergone this last review. Patients who have received at least 1 cycle of study agent are evaluable for safety and for clinical and biological response. Proportions and durations of progression-free survival are summarised by Kaplan-Meier methods. Toxicity is summarised according to Common Terminology Criteria for Adverse Events Version 3.0.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as broadly described herein.

Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1-34. (canceled)

35. A polypeptide comprising an estrogen or androgen binding region, the binding region capable of binding to an estrogen or androgen at a sufficient affinity or avidity such that upon administration of the polypeptide to a mammalian subject the level of biologically available estrogen or androgen is decreased.

36. A polypeptide according to claim 35, wherein the level of biologically available estrogen is measured in the blood, a breast cell or an ovarian cell of the subject; and wherein the level of biologically available androgen is measured in the blood or an endometrial cell of the subject.

37. A polypeptide according to according to claim 35, wherein the level of biologically available estrogen or androgen is decreased such that the growth of a breast cancer cell, an ovarian cancer cell or an endometrial cancer cell in the subject is decreased or substantially arrested.

38. A polypeptide according to according to claim 35, having an affinity or avidity for an estrogen or androgen that is equal to or greater than the affinity or avidity between the estrogen or the androgen and a protein that naturally binds to the estrogen or the androgen.

39. A polypeptide according to claim 35, wherein the estrogen binding region comprises the estrogen binding domain from the human estrogen receptor, or a functional equivalent thereof, and wherein the androgen binding region comprises the androgen binding domain from the human androgen receptor, or a functional equivalent thereof.

40. A polypeptide according to claim 35, wherein the estrogen or androgen binding region comprises the estrogen or androgen binding domain from sex hormone binding globulin, or a functional equivalent thereof.

41. A polypeptide according to claim 35, having a single estrogen or androgen binding region.

42. A polypeptide according to claim 35, comprising a carrier region.

43. A polypeptide according to claim 35, wherein the carrier region is the Fc region of human IgG, or a functional equivalent thereof.

44. A polypeptide according to claim 35, that is selected from the group consisting of a fusion protein, a monoclonal antibody, a polyclonal antibody, and a single chain antibody.

45. A nucleic acid molecule capable of encoding a polypeptide according to claim 35.

46. A vector comprising a nucleic acid molecule according to claim 45.

47. A composition comprising a polypeptide according to claim 35 and a pharmaceutically acceptable carrier.

48. A method for treating or preventing an estrogen-related cancer or an androgen-related cancer in a subject, the method comprising administering to a subject in need thereof an effective amount of a ligand capable of binding estrogen or androgen in the subject, or an effective amount of a nucleic acid molecule according to claim 45, or an effective amount of a vector according to claim 46 or an effective amount of a composition according to claim 47, such that the level of biologically available estrogen or androgen in the subject is decreased as compared with the level of biologically available estrogen or androgen present in the subject prior to administration of the ligand or the nucleic acid molecule or the vector.

49. A method according to claim 48, wherein the estrogen-related cancer is selected from the group consisting of breast cancer and ovarian cancer, and wherein the androgen-related cancer is endometrial cancer.

50. A method according to claim 48, wherein the level of biologically available estrogen is measured in the blood, a breast cell or an ovarian cell of the subject, and wherein the level of biologically available androgen is measured in the blood or an endometrial cell of the subject.

51. A method according to claim 48, wherein the ligand is a polypeptide according to claim 1.

52. A method for treating or preventing estrogen flare or testosterone flare in the treatment of a subject having estrogen-related cancer with an LHRH agonist or antagonist comprising administering to a subject in need thereof an effective amount of a polypeptide according to claim 35.
